(19) United States
(12) Patent Application Publication
(10) Pub. No.: US 2014/0100188 A1
(43) Pub. Date: Apr. 10, 2014

(54) PHENOTYPING TUMOR-INFILTRATING LEUKOCYTES

(71) Applicants: University College Dublin, Dublin (IE); The Regents of the University of California, Oakland, CA (US)

(72) Inventors: Lisa M. COUSSENS, Lake Oswego, OR (US); David G. DENARDO, Olivette, MO (US); Donal J. BRENNAN, Hamilton Island (AU)

(73) Assignees: University College Dublin, Dublin (IE); The Regents of the University of California, Oakland, CA (US)

(21) Appl. No.: 14/044,715

(22) Filed: Oct. 2, 2013

Related U.S. Application Data
(63) Continuation-in-part of application No. 13/314,072, filed on Dec. 7, 2011.
Application No. 13/314,072 is a continuation-in-part of application No. PCT/US2010/042654, filed on Jul. 20, 2010.
(60) Provisional application No. 61/744,839, filed on Oct. 3, 2012.
(60) Provisional application No. 61/420,718, filed on Dec. 7, 2010.
(60) Provisional application No. 61/227,035, filed on Jul. 20, 2009.

(51) Int. Cl. C12Q 1/68; G01N 33/574

(52) U.S. Cl. CPC C12Q 1/6886; G01N 33/57438
USPC 514/50; 435/7.24; 514/49; 435/6.12; 506/9; 514/266.4; 514/410

(57) ABSTRACT

The present disclosure relates to an immune signature of tumor infiltrating leukocytes. In particular, the disclosure provides methods and kits for determining the immune signature of tumor infiltrating leukocytes for use in assessing risk of cancer recurrence and long term survival, and for developing a treatment regimen for a cancer patient.

### STATEMENT OF GOVERNMENT SUPPORT

This invention was made with Government support under grant no. W81XWH-06-1-0416 awarded by the United States Army Medical Research and Materiel Command. The government has certain rights in the invention.

### FIELD

The present disclosure relates to an immune signature of tumor infiltrating leukocytes. In particular, the disclosure provides methods and kits for determining the immune signature of tumor infiltrating leukocytes for use in assessing risk of cancer recurrence and long term survival, and for developing a treatment regimen for a cancer patient.

### BACKGROUND

Cancer is a heterogeneous disease. There is widespread interest in identifying molecular characteristics of tumors indicative of their behavior and response to therapy in order to reduce morbidity and mortality. A better understanding of the molecular characteristics of tumors and their immune environment would also be valuable in reducing the use of toxic chemotherapy drugs on patients for whom these medicines would be ineffective and/or unnecessary for treating their cancers.

In the case of breast cancer, genome wide analyses of the associations between gene copy number (Bergamaschi et al., Genes Chromosomes Cancer, 45:1033-1040, 2006; and Chin et al., Cancer Cell, 10:529-541, 2006), gene expression (Sorlie et al., Proc Natl Acad Sci USA, 98:10869-10874, 2001; Perou et al., Nature, 406:747-752, 2000; Sorlie et al., Proc Natl Acad Sci USA, 100:8418-8423, 2003; van de Vijver et al., N Engl J Med, 347:1999-2009, 2002; van 't Veer, et al., Nature, 415:530-536, 2002; Rakha et al., Cancer, 109:25-32, 2007; Finnegan and Carey, Future Oncology, 3:55-63, 2007, and Cheang et al., Clin Cancer Res, 14:1368-1376, 2008), and clinical outcome have revealed two major breast cancer subtypes, “basal” and “luminal/amplifier,” which are associated with distant recurrence and short survival duration. Basal subtype tumors are referred to as “triple negatives” because they do not express the estrogen receptor (ER), the progesterone receptor (PR) or the human epidermal growth factor receptor 2 (HER2/nue or ErbB2). As such basal subtype tumors are not expected to be sensitive to anti-estrogen therapies or trastuzumab.

Currently, in the clinical setting, the standard of care for breast cancer is to evaluate breast cancer tissue for prognostic markers including estrogen receptor (ER), progesterone receptor (PR), and HER2 expression levels. Independently, these biomarkers are used to help guide treatment decisions and overall disease management. More recently, gene signature approaches have been optimized by several groups: 21-gene Oncotype DX (Genomic Health); 70-gene MammoPrint (Agendia); 76-gene “Rotterdam signature” (Veridex); and 41-gene signature (Ahr et al., J Pathol, 195:312-320, 2001). To varying degrees these approaches have been shown to have predictive value for improving risk assessment of patients with breast cancer. A significant percentage of patients however, develop recurrent disease despite having a tumor epithelial cell gene expression pattern that is predictive of a low risk of progression. Moreover, some patients having a tumor epithelial cell gene expression pattern that is predictive of a high risk of disease progression, undergo chemotherapy, but may still not have experienced the expected rapid rate of disease progression with a less aggressive course of therapy. Thus, the determination of the gene expression profile of cancer cells alone is not sufficient to fully assess risk.

In the United States, breast cancer and ovarian cancer are the first and eighth most prevalent cancers in women, and the second and fifth most common cause of cancer-related death in women (U.S. Cancer Statistics Working Group, United States Cancer Statistics: 1999-2006 Incidence and Mortality Web-based Report, Atlanta: U.S. Department of Health and Human Services, Center for Disease Control and Prevention, and National Cancer Institute). The poor ratio of survival to incidence of ovarian cancer is a consequence of late diagnosis and the lack of effective therapies for advanced refractory disease. Despite improvements in surgical techniques and the advent of more targeted therapeutics, survival of ovarian cancer patients is 45% at five years (Jemal et al., CA Cancer J Clin, 58:71-96, 2008). New tools to facilitate epithelial ovarian cancer diagnosis and patient stratification are therefore needed to improve the efficacy of available treatment options.

Current management of ovarian cancer involves initial surgical debulking, which incorporates total abdominal hysterectomy, bilateral salpingo-oophorectomy and omentectomy with cytological evaluation of peritoneal fluid or washings. One of the most significant predictors of patient outcome to date is the extent of residual disease after primary surgery (Bristow et al., J Clin Oncol, 20:1248-1259, 2002), and current clinical recommendations include complete debulking leaving no macroscopic residuals (Guarneri et al., Gynecol Oncol, 117:152-158, 2010). Adjuvant systemic chemotherapy for ovarian cancer is empiric and initial treatment involves paclitaxel-platinum-based regimens that continue to show improved outcomes compared to other cytotoxic agents such as gecitabine, topotecan and liposomal doxorubicin (Bookman et al., J Clin Oncol, 27:1419-1425, 2009). Despite aggressive surgery and chemotherapy, the majority of ovarian cancer patients relapse within 3-5 years and the median time to relapse is 15 months post diagnosis (Hennessy et al., Lancet, 374:1371-1382, 2009).

There are separate histological subgroups of epithelial ovarian cancer, including serous subgroups and non-serous subgroups (e.g., endometroid, clear cell and mucinous tumors), which are characterized by different clinical behaviors. Approximately 70% of tumors are serous and have a distinctly worse prognosis than other forms of ovarian cancer (Kobel et al., PLoS Med, 5:e232, 2008). Patient stratification according to histological subtypes is therefore desirable.

Ovarian cancer patients are further stratified based on their treatment-free interval after platinum-based chemotherapy (Markman and Hoskins, J Clin Oncol, 10:513-514, 1992). Patients were classified as having platinum-resistant ovarian cancer if progression occurred on primary platinum-based therapy, less than a partial response to a platinum-based regimen (Therasse et al., J Natl Cancer Inst, 92:205-216, 2000), or recurrence within six months of completing a platinum-based regimen. Alternatively, patients were classified as having platinum-sensitive ovarian cancer if they demonstrated at least a partial response to a platinum-based regimen and had a treatment-free interval of more than six months. This classification is particularly relevant in determining treatment regimens for recurrent disease as the probability of response to platinum-retreatment is closely related to the duration of platinum-free interval (Markman, Trends Pharmacol Sci, 29:515-519, 2008).

Clinical and experimental studies have established that chronic infiltration of neoplastic tissue by leukocytes (e.g., chronic inflammation) promotes development and/or progression of various epithelial tumors (de Visser et al., Nature Reviews Cancer 6, 24-37, 2006; and Mantovani et al., Nature, 454:436-444, 2008). The organ-specific cellular and molecular programs that favor pro-tumor, as opposed to anti-tumor immunity, however, are incompletely understood. While some subsets of leukocytes exhibit anti-tumor activity, including cytotoxic CD8⁺ T lymphocytes (CTLs) and natural killer (NK) cells (Dunn et al., Immunity, 21:137-148, 2004), other leukocytes exhibit more bipolar roles. Most notably, mast cells, CD4⁺ T lymphocytes, B lymphocytes, dendritic cells, granulocytes and macrophages, have the capacity to either hinder or potentiate tumor progression (Ostrand-Rosenberg, Curr Opin Genet Dev, 18:11-18, 2008; and de Visser et al., Cancer Cell, 7:411-423, 2005).

Several recent studies have analyzed the influence of host immunity on disease prognosis. Tumor-infiltrating CD3+ T cells are strongly associated with favorable prognosis (Zhang et al., N Engl J Med, 348:203-213, 2003; Raspollini et al., Ann Oncol, 16:590-596, 2005; and Nelson, Immnol Rev, 222:101-116, 2008), with a particular emphasis on the CD8+ cytotoxic T cell subset (Hamanishi et al., Proc Natl Acad Sci USA, 104:3360-3365, 2007; Sato et al., Proc Natl Acad Sci USA, 102:18538-18543, 2005; Clarke et al., Mod Pathol, 22:393-402, 2009; and Milne et al., PLoS One, 4:e6412, 2009), suggesting that cytotoxic T lymphocytes (CTLs) play an important role in the antitumor immune response. Accordingly, other factors associated with CTL responses are also associated with an improved prognosis, including interferon-γ (IFN-γ) (Marth et al., AM J Obstet Gynecol, 191:1598-1605; and Kusuda et al., Oncol Rep, 12:1153-1158, 2005), the IFN-γ receptor (Duncan et al., Clin Cancer Res, 13:4139-4145, 2007), TNFα (Kusuda, 2005, supra) and MHC class I (Rolland et al., Clin Cancer Res, 13:3591-3596, 2007; and Leffers et al., Gynecol Oncol, 110:365-373, 2008).

Since tumor growth and development is influenced by the phenotype of the immune response to neoplasia, as well as the genotype of malignant cells, there is a need for tools to characterize a patient's immune response to their tumor. Additional patient stratification criteria would provide valuable guidance for treatment regimen selection.

### SUMMARY

The present disclosure relates to an immune signature of tumor infiltrating leukocytes. In particular, the disclosure provides methods and kits for determining the immune signature of tumor infiltrating leukocytes for use in assessing risk of cancer recurrence and long term survival, and for developing a treatment regimen for a cancer patient.

The present disclosure provides methods for assessing risk of poor clinical outcome for a human cancer patient, the method comprising: a) subjecting a tumor sample from the patient to a procedure for quantitation of expression of leukocyte biomarkers comprising CD4, CD8 and CD68; and b) detecting the presence of an immune signature of poor outcome comprising CD4^{hi}/CD8^{lo}/CD68^{hi} or an immune signature of favorable outcome comprising CD4^{lo}/CD8^{hi}/CD68^{lo} in the tumor sample, wherein the immune signature of poor outcome is associated with an increased risk of poor clinical outcome as compared to the immune signature of favorable outcome. In other embodiments, the present disclosure provides methods for assessing risk of poor clinical outcome for a human cancer patient, the method comprising: a) subjecting a tumor sample from the patient to a procedure for quantitation of expression of leukocyte biomarkers comprising CD8 and CD68; and b) detecting the presence of an immune signature of poor outcome comprising CD8^{lo}/CD68^{hi} or an immune signature of favorable outcome comprising CD8^{hi}/CD68^{lo} in the tumor sample, wherein the immune signature of poor outcome is associated with an increased risk of poor clinical outcome as compared to the immune signature of favorable outcome, and wherein the biomarkers do not comprise CD4. In some embodiments, the leukocyte biomarkers consist essentially of CD8 and CD68. In some preferred embodiments, the sample is from a solid tumor. In some embodiments, the procedure for quantitation comprises an antibody-based technique. In some preferred embodiments, the antibody-based technique comprises a procedure selected from but not limited to immunohistochemistry, flow cytometry, antibody microarray, ELISA, western blotting, and magnetic resonance imaging. In other embodiments, the procedure for quantitation comprises a nucleic-acid based technique. In some preferred embodiments, the nucleic acid based technique comprises a procedure selected from but not limited to RT-PCR, nucleic acid microarray, serial analysis of gene expression, massively parallel signature sequencing, in situ hybridization, and northern blotting. In some embodiments, the poor clinical outcome comprises a relative reduction in one or more of overall survival, recurrence-free survival (cancer relapse), and distant recurrence-free survival (cancer metastasis). In some embodiments, the methods further comprise: c) treating the patient with an aggressive treatment regimen when the immune signature of poor outcome is detected. In some embodiments, the favorable clinical outcome comprises a relative increase in one or more of overall survival, recurrence-free survival, and distant recurrence-free survival. In some embodiments, the methods further comprise detecting metastasis to a regional or a draining lymph node of the human cancer patient. In some embodiments, the methods further comprise: c) treating the patient with a conservative treatment regimen when the immune signature of favorable outcome is detected. In some embodiments, the methods further comprise: c) treating the patient with a platinum-based chemotherapy drug when the immune signature of favorable outcome is detected (e.g., indicative of a platinum-sensitive tumor). In some embodiments, the methods further comprise: c) treating the patient with a taxane-based chemotherapy drug (e.g., paclitaxel, docetaxel, etc.) when the immune signature of poor outcome is detected (e.g., indicative of a platinum-resistant tumor). In some embodiments, the tumor sample is from a breast cancer biopsy, lumpectomy or resection. In other embodiments, the tumor sample is selected from but not limited to a breast (e.g., invasive ductal carcinoma, invasive lobular carcinoma, ductal carcinoma in situ, and lobular carcinoma in situ), bladder, cervical, colon, lung, mouth, ovarian, prostate, rectal, renal, testicular, and uterine cancer biopsy. In some embodiments, the methods further comprise determining subtype of the solid tumor. In some embodiments, when the solid tumor is breast cancer, the subtype is selected from the group consisting of basal, luminal A, luminal B, and triple negative. In some embodiments, when the solid tumor is breast cancer, the subtype is selected from the group consisting of HER2+ and basal. In some embodiments, the ovarian cancer is epithelial ovarian cancer of a serous subtype. In other embodiments, the ovarian cancer is epithelial ovarian cancer of a non-serous subtype. In some embodiments, the methods further comprise: c) treating the patient with a regimen suitable for a basal or luminal A subtype of breast cancer. In some embodiments, the methods further comprise: c) treating the patient with a regimen suitable for a serous type of epithelial ovarian cancer. In some embodiments, the methods further comprise: c) treating the patient with an immune modulator. In some embodiments, the immune modulator is an anti-Th2 or a pro-Th1 immune modulator. In some embodiments, the immune modulator is an inhibitor of colony stimulating factor 1 (CSF1, also known as monocyte colony stimulator factor, or M-CSF) or its receptor. In additional embodiments, the methods further comprise one or both steps before a) of obtaining the tumor sample from the patient, and establishing a cut-off value for distinguishing between high and low expression of the leukocyte biomarkers.

Moreover the present disclosure provides methods for assessing risk of poor clinical outcome for a human breast cancer or ovarian cancer patient, the method comprising: a) subjecting a breast cancer or epithelial ovarian cancer sample from the patient to an antibody-based technique for quantitation of expression of leukocyte biomarkers comprising CD4, CD8 and CD68; and b) detecting the presence of an immune signature of poor outcome comprising CD4^{hi}/CD8^{lo}/CD68^{hi} or an immune signature of favorable outcome comprising CD4^{lo}/CD8^{hi}/CD68^{lo} in the sample, wherein the immune signature of poor outcome is associated with an increased risk of poor clinical outcome as compared to the immune signature of favorable outcome. In additional embodiments, the method comprises: a) subjecting a breast cancer or epithelial ovarian cancer sample from a patient to an antibody-based technique for quantitation of expression of leukocyte biomarkers comprising CD8 and CD68; and b) detecting the presence of an immune signature of poor outcome comprising CD8^{lo}/CD68^{hi} or an immune signature of favorable outcome comprising CD8^{hi}/CD68^{lo} in the sample, wherein the immune signature of poor outcome is associated with an increased risk of poor clinical outcome as compared to the immune signature of favorable outcome. In further embodiments, the method comprises: a) subjecting a pancreatic cancer sample from a patient to an antibody-based technique for quantitation of expression of leukocyte biomarkers comprising CD8 and CD68; and b) detecting the presence of an immune signature of poor outcome comprising CD8^{lo}/CD68^{hi} or an immune signature of favorable outcome comprising CD8^{hi}/CD68^{lo} in the sample, wherein the immune signature of poor outcome is associated with an increased risk of poor clinical outcome as compared to the immune signature of favorable outcome. In some embodiments, the biomarkers do not comprise CD4. In some embodiments, the pancreatic cancer is pancreatic ductal adenocarcinoma (PDAC). In some embodiments, the biomarkers do not comprise CD4. In some embodiments, the leukocyte biomarkers consist essentially of CD8 and CD68. In some embodiments, the antibody-based technique comprises immunohistochemistry. In some embodiments, the poor clinical outcome comprises a relative reduction in one or more of overall survival, recurrence-free survival (cancer relapse), and distant recurrence-free survival (cancer metastasis). In some embodiments, the methods further comprise detecting metastasis to a regional or a draining lymph node of the human cancer patient. In some preferred embodiments, the methods further comprise: c) treating the patient with an aggressive treatment regimen when the immune signature of poor outcome is detected. In some embodiments, the methods further comprise: c) treating the patient with a palliative treatment regimen when the immune signature of poor outcome is detected. In some embodiments in which the patient is a pancreatic cancer patient, the palliative treatment regimen comprises a surgical resection with palliative intent selected from the group consisting gastric bypass, biliary bypass, and biliary stent. In other embodiments, the favorable clinical outcome comprises a relative increase in one or more of overall survival, recurrence-free survival, and distant recurrence-free survival. In some embodiments, the breast cancer subtype is selected from the group consisting of basal, luminal A, and triple negative. In some embodiments, the breast cancer subtype is selected from the group consisting of HER2+ and basal. In some embodiments, the epithelial ovarian cancer of a serous subtype. In other embodiments, the epithelial ovarian cancer of a non-serous subtype. In some preferred embodiments, the method further comprises: c) treating the patient with a conservative treatment regimen when the immune signature of favorable outcome is detected. In some embodiments, the methods further comprise: c) treating the patient with a regimen suitable for a basal or luminal A subtype of breast cancer. In some embodiments, the methods further comprise: c) treating the patient with a regimen suitable for a serous type of epithelial ovarian cancer. In some embodiments, the methods further comprise: c) treating the patient with an immune modulator. In some embodiments, the immune modulator is an anti-Th2 or a pro-Th1 immune modulator. In some embodiments, the immune modulator is an inhibitor of colony stimulating factor 1 (CSF1, also known as monocyte colony stimulator factor, or M-CSF) or its receptor. In some embodiments, the methods further comprise: c) treating the patient with a curative treatment regimen when the immune signature of favorable outcome is detected. In some embodiments in which the patient is a pancreatic cancer patient, the curative treatment regimen comprises a radical pancreatic resection (e.g., Whipple procedure, total pancreatectomy and distal pancreatectomy). In some embodiments, the methods further comprise post-operative chemotherapy comprising one or more of gemcitabine, fluorouracil, erlotinib and mitomycin c. In additional embodiments, the methods further comprise one or both steps before a) of obtaining the sample from the patient, and establishing a cut-off value for distinguishing between high and low expression of the leukocyte biomarkers.

Moreover the present disclosure provides methods for assessing risk of poor clinical outcome for a human breast cancer or ovarian cancer patient, the method comprising: a) subjecting a breast cancer or epithelial ovarian cancer sample from the patient to a nucleic acid-based technique for quantitation of expression of leukocyte biomarkers comprising CD4, CD8 and CD68; and b) detecting the presence of an immune signature of poor outcome comprising CD4^{hi}/CD8^{lo}/CD68^{hi} or an immune signature of favorable outcome comprising CD4^{lo}/CD8^{hi}/CD68^{lo} in the sample, wherein the immune signature of poor outcome is associated with an increased risk of poor clinical outcome as compared to the immune signature of favorable outcome. In additional embodiments, the method comprises: a) subjecting a breast cancer or epithelial ovarian cancer sample from a patient to a nucleic acid-based technique for quantitation of expression of leukocyte biomarkers comprising CD8 and CD68; and b) detecting the presence of an immune signature of poor outcome comprising CD8^{lo}/CD68^{hi} or an immune signature of favorable outcome comprising CD8^{hi}/CD68^{lo} in the sample, wherein the immune signature of poor outcome is associated with an increased risk of poor clinical outcome as compared to the immune signature of favorable outcome. In further embodiments, the method comprises: a) subjecting a pancreatic cancer sample from a patient to a nucleic acid-based technique for quantitation of expression of leukocyte biomarkers comprising CD8 and CD68; and b) detecting the presence of an immune signature of poor outcome comprising CD8^{lo}/CD68^{hi} or an immune signature of favorable outcome comprising CD8^{hi}/CD68^{lo} in the sample, wherein the immune signature of poor outcome is associated with an increased risk of poor clinical outcome as compared to the immune signature of favorable outcome. In some embodiments, the pancreatic cancer is a pancreatic adenocarcinoma (PDAC). In some embodiments, the biomarkers do not comprise CD4. In some embodiments, the leukocyte biomarkers consist essentially of CD8 and CD68. In some embodiments, the nucleic acid-based technique comprises reverse transcriptase-polymerase chain reaction or nucleic acid microarray. In some embodiments, the poor clinical outcome comprises a relative reduction in one or more of overall survival, recurrence-free survival (cancer relapse), and distant recurrence-free survival (cancer metastasis). In some embodiments, the methods further comprise detecting metastasis to a regional or a draining lymph node of the human cancer patient. In some preferred embodiments, the methods further comprise: c) treating the patient with an aggressive treatment regimen when the immune signature of poor outcome is detected. In some embodiments, the methods further comprise: c) treating the patient with a palliative treatment regimen when the immune signature of poor outcome is detected. In some embodiments in which the patient is a pancreatic cancer patient, the palliative treatment regimen comprises a surgical resection with palliative intent selected from the group consisting gastric bypass, biliary bypass, and biliary stent. In other embodiments, the favorable clinical outcome comprises a relative increase in one or more of overall survival, recurrence-free survival, and distant recurrence-free survival. In some embodiments, the breast cancer subtype is selected from the group consisting of basal, luminal A, and triple negative. In some embodiments, the breast cancer subtype is selected from the group consisting of HER2+ and basal. In some embodiments, the epithelial ovarian cancer of a serous subtype. In other embodiments, the epithelial ovarian cancer of a non-serous subtype. In some preferred embodiments, the method further comprises: c) treating the patient with a conservative treatment regimen when the immune signature of favorable outcome is detected. In some embodiments, the methods further comprise: c) treating the patient with a regimen suitable for a basal or luminal A subtype of breast cancer. In some embodiments, the methods further comprise: c) treating the patient with a regimen suitable for a serous type of epithelial ovarian cancer. In some embodiments, the methods further comprise: c) treating the patient with an immune modulator. In some embodiments, the immune modulator is an anti-Th2 or a pro-Th1 immune modulator. In some embodiments, the immune modulator is an inhibitor of colony stimulating factor 1 (CSF1, also known as monocyte colony stimulator factor, or M-CSF) or its receptor. In some embodiments, the methods further comprise: c) treating the patient with a curative treatment regimen when the immune signature of favorable outcome is detected. In some embodiments in which the patient is a pancreatic cancer patient, the curative treatment regimen comprises a radical pancreatic resection (e.g., Whipple procedure, total pancreatectomy and distal pancreatectomy). In some embodiments, the methods further comprise post-operative chemotherapy comprising one or more of gemcitabine, fluorouracil, erlotinib and mitomycin c. In additional embodiments, the methods further comprise one or both steps before a) of obtaining the sample from the patient, and establishing a cut-off value for distinguishing between high and low expression of the leukocyte biomarkers. In addition, the present disclosure provides kits for assessing risk of poor clinical outcome for a human cancer patient, the kit comprising biomarker-specific reagents consisting essentially of: a) a CD4-specific reagent; b) a CD8-specific reagent; and c) a CD68-specific reagent. In some preferred embodiments, the CD4-specific reagent, the CD8-specific reagent and the CD68-specific reagent are antibodies. In other preferred embodiments, the CD4-specific reagent, the CD8-specific reagent and the CD68-specific reagent are nucleic acids. In some embodiments, the kits further comprise instructions for assessing risk of poor clinical outcome according to the methods of the preceding paragraphs.

In further embodiments, the present disclosure provides kits for assessing risk of poor clinical outcome for a human cancer patient, the kit comprising biomarker-specific reagents consisting essentially of: a) a CD8-specific reagent; and b) a CD68-specific reagent. In some preferred embodiments, the CD8-specific reagent and the CD68-specific reagent are antibodies. In other preferred embodiments, the CD8-specific reagent and the CD68-specific reagent are nucleic acids. In some embodiments, the kits further comprise instructions for assessing risk of poor clinical outcome according to the methods of the preceding paragraphs.

### BRIEF DESCRIPTION OF THE DRAWINGS

FIG. 1 illustrated that a CD68/CD4/CD8 immune-based signature is a significant independent predictor of patient survival. A-C) Representative high power images (40×) with 60× inlays of representative human breast cancer specimens showing expression of CD68⁺ (A), CD4⁺ (B) and CD8⁺ (C). D-F) Automated analysis of CD68⁺ (D), CD4⁺ (E) and CD8⁺ (F) immuno-detection reveals a relationship between leukocyte density and overall survival. Kaplan-Meier estimate of overall survival comparing autoscore leukocyte high and low infiltration groups is shown. 179 samples were used in all analyses and log rank (mantel-cox) p values are denoted for difference in overall survival. G-H) A Kaplan-Meier estimate of overall survival comparing CD68^{hi}/CD4^{hi}/CD8^{lo} and CD68^{lo}/CD4^{lo}/CD8^{hi} immune profiles as assigned by random forest clustering, to identify optimum thresholds in Cohort I (179 samples). These results were validated in Cohort II (498 samples). In (G) and (H) the log rank (mantel-cox) p value is denoted for the observed difference in overall survival in Cohort I and Cohort II respectively. I) A diagram illustrating the development of the CD68/CD4/CD8 signature. Regression tree analysis was used to define the signature based on continuous immune cell density data. All cases were defined as CD68^{hi}/CD4^{hi}/CD8^{lo} or CD68^{lo}/CD4^{lo}/CD8^{hi}. The signature was defined in Cohort I, and validated using the same thresholds in Cohort II.

FIG. 2 illustrates that a CD68/CD4/CD8 immune-based signature predicts patient survival independently. A-B) A Kaplan-Meier estimate of overall survival is shown comparing CD68^{hi}/CD4^{hi}/CD8^{lo} and CD68^{lo}/CD4^{lo}/CD8^{hi} immune profiles for Luminal A and Basal tumor subtypes in Cohort II.

FIG. 3 illustrates that a CD68/CD4/CD8 immune-based signature is a significant independent predictor of recurrence-free survival in ovarian cancer. A-C) Representative high power images (20×) are provided of representative human ovarian cancer specimens showing expression of CD4⁺ (A), CD68⁺ (B), and CD8⁺ (C). D-F) The automated analysis of CD4⁺ (D), CD68⁺ (E), and CD8⁺ (F) immuno-detection reveals a relationship between leukocyte density and overall survival. A Kaplan-Meier estimate of recurrence-free survival comparing autoscore leukocyte high and low infiltration groups is shown. 76 samples were used in all analyses and log rank (mantel-cox) p values are denoted for the difference in overall survival.

FIG. 4 illustrates that a CD68/CD4/CD8 immune-based signature is a significant independent predictor of recurrence-free survival in ovarian cancer. A) A Kaplan-Meier estimate of overall survival comparing CD68^{hi}/CD4^{hi}/CD8^{lo} and CD68^{lo}/CD4^{lo}/CD8^{hi} immune profiles as assigned by decision tree analysis and 10-fold cross validation was employed. 76 samples were assessed; the log rank (mantel-cox) p value is denoted for difference in overall survival. H) Results from multivariate Cox regression analysis are provided for a 3-marker immune based “signature” considering tumor stage, grade, patient's age at diagnosis and residual disease after primary surgery.

FIG. 5 illustrates that a CD68/CD4/CD8 immune-based signature is a predictor of recurrence-free survival in serous ovarian cancer tumors. A-B) A Kaplan-Meier estimate of overall survival comparing CD68^{hi}/CD4^{hi}/CD8^{lo} and CD68^{lo}/CD4^{lo}/CD8^{hi} immune profiles as assigned by decision tree analysis and 10-fold cross validation was employed to identify optimum thresholds in non-serous tumors (n=23) (A) and in serous tumors (n=53) (B).

FIG. 6 illustrates that immune infiltrates are different in serous compared to non-serous ovarian tumors. A-D) Analysis of mean cell density for CD68⁺ (A), CD20⁺ (B), CD8⁺ (C), and CD4⁺ (D). Error bars represent 2 SEM. * P<0.05.

FIG. 7 illustrates that the ratio of CD68 to CD8 predicts patient survival and response to neo-adjuvant chemotherapy. A) Kaplan-Meier estimate of survival comparing CD68^{high}/CD8^{low} and CD68^{low}/CD8^{high} immune profiles as assessed by mRNA expression from 3,872 samples across 14 different platforms is depicted. Median expression for both CD8 and CD68 was used to determine high and low groups within each of the 22 individual datasets. Once a sample was assigned to a particular group the 22 datasets were combined and a global survival analysis was performed. The log rank (Mantel-Cox) p-value is denoted for difference in survival. B) Analysis of pathologic complete remission (pCR) in frequency in patients in a cohort of 311 patients constructed from two independent data sets. All patients had fine needle aspirates (FNA) taken prior to neoadjuvant chemotherapy and pathological response was assessed at the time of definitive surgery. Using median expression as a threshold, examination of CD8 and CD68 mRNA in FNA samples demonstrated three specific groups: CD68>CD8, CD68<CD8, and CD68=CD8 (denoted CD68^{high}/CD8^{low}, CD68/CD8^{equal}, and CD68^{low}/CD8^{high} respectively). Analysis of the rate of pCR in these groups is depicted.

FIG. 8 illustrates that the ratio of CD68 to CD8 predicts patient survival in HER2+ and basal breast tumors. A-B) Kaplan-Meier estimates of survival comparing CD68^{high}/CD8^{low} and CD68^{low}/CD8^{high} immune profiles as assessed by mRNA expression from 3,872 patient samples across 14 different platforms is depicted. Median expression for both CD8 and CD68 was used to determine high and low groups within each of the 22 individual datasets. Once a sample was assigned to a particular group, the 22 datasets were combined and a global survival analysis was performed. The log rank (Mantel-Cox) p-value is denoted for difference in survival for Basal and HER2+(A) and basal (B) tumor subtypes.

FIG. 9 illustrates that the CD68/CD8 immune-profile signature is an independent prognostic indicator of overall survival in breast cancer patients. A-B) Kaplan-Meier estimates of overall survival (OS) comparing CD68^{high}/CD8^{low} and CD68^{low}/CD8^{high} immune profiles as assigned by random forest clustering employed to identify optimum thresholds using Cohort I. In addition, CD68^{high}/CD8^{low} and CD68^{low}/CD8^{high} immune profiles were employed to stratify a second independent Cohort II. The log rank (Mantel-Cox) p-value is denoted for difference in OS in (A) Cohort I (n=179) and (B) Cohort II (n=498).

FIG. 10 illustrates that the ratio of CD68 to CD8 predicts overall survival (OS) of pancreatic ductal adenocarcinoma (PDAC) patients. A Kaplan-Meier estimate of survival comparing CD68^{high}/CD8^{low} and CD68^{low}/CD8^{high} immune profiles as assessed by immune detection of 59 samples is depicted. The log-rank (Mantel-Cox) p-value is denoted.

### DETAILED DESCRIPTION

A three-marker (CD4, CD8 and CD68) and a two-marker (CD8 and CD68) immune-based profile is provided to robustly evaluate a cancer patient's immune response to malignancy at the time of biopsy and/or surgery. Characterization of tumor-infiltrating leukocytes permits predictions to be made regarding clinical outcome and likelihood of progression and/or relapse. As immune signatures are not apparently linked to the molecular and genetic heterogeneity of the tumor subtype; assessing immune responses to solid tumors provides an additional dimension to existing gene expression-based prognostic tools, which heretofore were solely reliant on the underlying tumor cell genetic and epigenetic features.

### Prognostic Methods

In one embodiment, cancer patients with cancer samples with a CD68^{hi}/CD4^{hi}/CD8^{lo} immune profile are identified as being at a greater risk for cancer metastasis and/or relapse, and as having a reduced overall survival rate or rate of recurrence-free survival, as compared to cancer patients having cancer samples without a CD68^{hi}/CD4^{hi}/CD8^{lo} immune profile. In another embodiment, cancer patients with cancer samples with a CD68^{lo}/CD4^{lo}/CD8^{hi} immune profile are identified as being at a reduced risk for cancer metastasis and/or relapse, and as having a greater overall survival rate or rate of recurrence-free survival, as compared to cancer patients having cancer samples without a CD68^{lo}/CD4^{lo}/CD8^{hi} immune profile.

In an additional embodiment, cancer patients with cancer samples with a CD68^{hi}/CD8^{lo} immune profile are identified as being at a greater risk for cancer metastasis and/or relapse, and as having a reduced overall survival rate or rate of recurrence-free survival, as compared to cancer patients having cancer samples without a CD68^{hi}/CD8^{lo} immune profile. In another embodiment, cancer patients with cancer samples with a CD68^{lo}/CD8^{hi} immune profile are identified as being at a reduced risk for cancer metastasis and/or relapse, and as having a greater overall survival rate or rate of recurrence-free survival, as compared to cancer patients having cancer samples without a CD68^{lo}/CD8^{hi} immune profile.

In one embodiment, threshold levels of each marker are established to define a ‘high’ or ‘low’ level of expression of the marker. Depending on the cancer type analyzed, the technique used and the marker examined, different values may be used to define a ‘high’ or ‘low’ level of expression of the marker. In order to define ‘high’ or ‘low’ levels of expression of a marker, statistical analysis such as random forest clustering may be used in order to identify optimum threshold levels.

A. Antibody Based Methods

In some embodiments, CD4, CD8, and CD68 levels are determined by using antibody-based methods to determine the levels of each biomarker protein in the tumor sample. Antibody-based methods include various techniques that involve the recognition of CD4, CD8, and CD68 antigens using specific antibodies. For most techniques, monoclonal antibodies are used. However, for some techniques polyclonal antibodies can be used. Commonly used antibody-based techniques to detect the level of one or more proteins in a sample include immunohistochemistry, flow cytometry, antibody microarray, ELISA, western blotting, and magnetic resonance imaging.

Immunohistochemistry.

Immunohistochemistry is the general process of determining the location and/or approximate level of one or more antigens in a tissue sample using antibodies directed against the antigens of interest. Typically, a thin slice of tumor tissue sample is cut from a larger tumor sample and mounted onto a slide, followed by treating the slice of tumor tissue with one or more reagents (including antibodies) to detect the antigens of interest. Immunohistochemistry can also be performed on tissue slices that are not mounted on a slide. In some instances, formalin-fixed and/or paraffin-embedded tissue samples are used for immunohistochemistry. Paraffinized samples can also be deparaffinized in order retrieve antigenicity of proteins.

During immunohistochemistry, antibody-antigen interactions can be detected through various mechanisms, including conjugating the antibody to an enzyme that can catalyze a color-producing reaction, such as a peroxidase, or conjugating the antibody to a fluorophore. A fluorophore is a molecule that will absorb energy at a specific wavelength and release energy at a different specific wavelength, e.g. fluorescein. The typical immunohistochemistry process involves treating first treating the thin tissue sample with blocking solution to reduce nonspecific background staining, followed by exposing the tissue sample the antibody or antibodies of interest, washing the tissue sample, and then visualizing the antibody-antigen complexes of interest.

Flow Cytometry.

To analyze tumor samples by flow cytometry, the tumor sample is processed to separate the tumor into individual cells. The cells are incubated with fluorophore-tagged antibodies of interest, and the collection of cells is processed through a flow cytometer. The flow cytometer uses different wavelengths of light to excite and detect different fluorophores. By analyzing a collection of cells from a tumor sample, which have been incubated with fluorophore-tagged antibodies of interest, a measurement of level of the different antigens of interest in the tumor sample can be obtained.

Antibody Microarray.

The general process for an antibody microarray is to bind a collection of antibodies against antigens of interest to a fixed surface (to create the microarray), to incubate the microarray with a sample that may contain the antigen(s) of interest, and then to add one or more reagents that allow for the detection of antibody microarray-bound antigens of interest. For antibody microarray analysis, a tumor sample is prepared by a homogenization technique which eliminates large tumor particles which could interfere with the function of the antibody microarray, but which preserves the integrity of the antigens of interest. Reagents that can be used for detection of antibody microarray-bound antigens of interest include fluorophore or enzyme-tagged antibodies.

Enzyme-Linked Immunosorbent Assay (ELISA).

To detect protein levels in a sample by ELISA, what is commonly known as a ‘Sandwich ELISA’ is performed. For a Sandwich ELISA, antibodies against an antigen of interest are linked to a surface. The surface-linked antibodies are exposed to a non-specific blocking agent, and then they are incubated with a sample containing the antigens of interest (e.g. in this case, a tumor sample). After incubation, the antibodies are washed to remove unbound material, and then antibodies, which bind to the antigen are added. These antibodies can be directly linked to a fluorophore or an enzyme to allow for their detection, or a secondary antibody linked to a fluorophore or an enzyme can be used to detect these antibodies. Through this technique, the level of one or more antigens in a sample can be determined.

Western Blotting.

For western blotting, a tumor sample of interest is homogenized, and a sample of the tumor is separated by polyacrylamide gel electrophoresis. The electrophoresis step separates proteins in the sample applied to the gel, and the proteins in the gel are next transferred to a membrane. Typically, PVDF or nitrocellulose membranes are used. After transferring proteins from the gel to the membrane, the membrane is treated with a non-specific blocking agent, and then incubated with antibodies against an antigen of interest. After incubation of the sample with the specific antibodies, the membrane is washed, and then treated with a secondary antibody, which binds to the specific antibody. The secondary antibody is typically linked to an enzyme, which can be used to create a reaction to detect the location and approximate level of the antigen of interest on the membrane.

Molecular Resonance Imaging.

Levels of proteins in a tumor inside a person's body can be determined through the use of specific molecular resonance imaging probes. Probes include antibodies directed against antigens of interest in the tumor, which are linked to molecules, which can be visualized in the body. Through the use of these techniques, the level of one or more proteins of interest in a tumor inside the body can be determined.

B. Nucleic Acid-Based Methods

In other embodiments, CD4, CD8, and CD68 levels are determined by using nucleic acid-based methods to determine the levels of each biomarker mRNA in the tumor sample. In general, methods of mRNA level and gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker and Barnes, Methods in Molecular Biology, 106:247-283, 1999); RNAse protection assays (Hod, Biotechniques, 13:852-854, 1992); and quantitative or semi-quantitative reverse transcription polymerase chain reaction (RT-PCR) (Weis et al., Trends in Genetics, 8:263-264, 1992). Representative methods for sequencing-based gene expression analysis include Serial Analysis of Gene Expression (SAGE), and gene expression analysis by massively parallel signature sequencing (MPSS).

RT-PCR.

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons, 1997. Methods for RNA extraction from paraffin embedded tissues are disclosed, for example, in Rupp and Locker, Lab Invest, 56:A, 1987, and De Andrés et al., BioTechniques, 18:42044, 1995. In particular, RNA isolation can be performed using purification kit, buffer set and protease from commercial manufacturers, such as Qiagen, according to the manufacturer's instructions. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™ The system consists of a thermocycler, laser, charge-coupled device (CCD), camera and computer. The system amplifies samples in a 96-well format on a thermocycler. During amplification, laser-induced fluorescent signal is detected at the CCD. The system includes software for running the instrument and for analyzing the data.

5′-Nuclease assay data are initially expressed as CT, or the threshold cycle. As discussed above, fluorescence values are recorded during every cycle and represent the amount of product amplified to that point in the amplification reaction. The point when the fluorescent signal is first recorded as statistically significant is the threshold cycle (C_{T}).

To minimize errors and the effect of sample-to-sample variation, RT-PCR is usually performed using one or more reference genes as internal standards. The ideal internal standard is expressed at a constant level among different tissues. RNAs most frequently used to normalize patterns of gene expression are mRNAs for the housekeeping genes glyceraldehyde-3-phosphate-dehydrogenase (GAPDH) and β-actin (ACTB).

A more recent variation of the RT-PCR technique is the real time quantitative PCR, which measures PCR product accumulation through a dual-labeled fluorogenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research, 6:986-994, 1996.

The steps of a representative protocol for profiling gene expression using fixed, paraffin-embedded tissues as the RNA source, including mRNA isolation, purification, primer extension and amplification are given in various published journal articles for example: Godfrey et al., J Molec Diagnostics, 2:84-91, 2000; Specht et al., Am J Pathol, 158:419-29, 2001; and Cronin et al., Am J Pathol, 164:35-42, 2004. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

Microarrays.

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, the microarrayed genes are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA, 93:106-149, 1996). Microarray analysis can be performed by commercially available equipment, following manufacturer's protocols, such as by using the Affymetrix GenChip technology, or Incyte's microarray technology.

Serial Analysis of Gene Expression (SAGE).

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

Gene Expression Analysis by Massively Parallel Signature Sequencing (MPSS)

This method, described by Brenner et al., Nature Biotechnology, 18:630-634, 2000, is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

General Description of the mRNA Isolation, Purification and Amplification.

The steps of a representative protocol for profiling gene expression using fixed, paraffin-embedded tissues as the RNA source, including mRNA isolation, purification, primer extension and amplification are provided in various published journal articles (e.g., Godfrey et al. J Molec Diagnostics, 2:84-91, 2000; and Specht et al., Am J Pathol, 158:419-29, 2001). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR. Finally, the data are analyzed to identify the best treatment option(s) available to the patient on the basis of the characteristic gene expression pattern identified in the tumor sample examined, dependent on the predicted likelihood of cancer recurrence.

Treatment Methods

In yet another embodiment, determination of CD4, CD8, and CD68 levels in a cancer patient is used to determine an optimal cancer treatment regimen for the patient. For a cancer patient with a cancer sample with a CD68^{hi}/CD4^{hi}/CD8^{lo} immune profile, an aggressive cancer treatment regimen may be indicated. In an additional embodiment, determination of CD8 and CD68 levels in a cancer patient is used to determine an optimal cancer treatment regimen for the patient. For a cancer patient with a cancer sample with a CD68^{hi}/CD8^{lo} immune profile, an aggressive cancer treatment regimen may be indicated. Aggressive cancer treatment regimens include but are not limited to local surgical resection of regional tumor tissue, and adjuvant systemic therapies. Surgical resection of regional disease includes lumpectomy, modified mastectomy or total mastectomy. Adjuvant systemic therapies include external radiation, combinatorial chemotherapy (for example six cycles of fluorouracil, doxorubicin and cyclophosphamide), as well as hormone and/or growth factor targeted therapy for patients with ER, PR or HER2 positive disease (e.g., tamoxifen or trastuzumab). For a cancer patient with a cancer sample with a CD68^{lo}/CD4^{lo}/CD8^{hi} immune profile, a conservative cancer treatment regimen may be indicated. Additionally, for a cancer patient with a cancer sample with a CD68^{lo}/CD8^{hi} immune profile, a conservative cancer treatment regimen may be indicated. Conservative cancer treatment regimens include but are not limited to local surgical resection and hormonal therapy, and would be similar to patients with low risk or Stage I disease with no lymph node involvement.

TABLE A
Risk Categories for Women with Node-Negative Breast Cancer

| | Low Risk | Intermediate Risk | High Risk |
|---|---|---|---|
| Tumor size | ≦1 cm | 1-2 cm | >2 cm |
| ER or PR Status | Positive | Positive | negative |
| Tumor grade | grade 1 | grade 1-2 | grade 2-3 |

TABLE B
Adjuvant Systemic Treatment Options for Women
with Axillary Node-Negative Breast Cancer

| Patient Group | Treatment |
|---|---|
| Low Risk | |
| Premenopausal, | None or tamoxifen |
| ER(+) or PR(+) | |
| Premenopausal, | — |
| ER(−) or PR(−) | |
| Postmenopausal, | None or tamoxifen |
| ER(+) or PR(+) | |
| Postmenopausal, | — |
| ER(−) or PR(−) | |
| Older than | None or tamoxifen |
| 70 years | |
| Intermediate Risk | |
| Premenopausal, | Tamoxifen plus chemotherapy, tamoxifen alone, |
| ER(+) or PR(+) | ovarian ablation, GnRH analog* |
| Premenopausal, | — |
| ER(−) or PR(−) | |
| Postmenopausal, | Tamoxifen plus chemotherapy, tamoxifen alone |
| ER(+) or PR(+) | |
| Postmenopausal, | — |
| ER(−) or PR(−) | |
| Older than | Tamoxifen alone, tamoxifen plus chemotherapy |
| 70 years | |
| High Risk | |
| Premenopausal, | Chemotherapy plus tamoxifen, chemotherapy plus |
| ER(+) or PR(+) | ablation or GnRH analog*, chemotherapy plus |
| | tamoxifen plus ovarian ablation or GnRH*, |
| | or ovarian ablation alone or with tamoxifen or |
| | GnRH alone or with tamoxifen |
| Premenopausal, | Chemotherapy |
| ER(−) or PR(−) | |
| Postmenopausal, | Tamoxifen plus chemotherapy, tamoxifen alone |
| ER(+) or PR(+) | |
| Postmenopausal, | Chemotherapy |
| ER(−) or PR(−) | |
| Older than | Tamoxifen; consider chemotherapy if ER-negative or |
| 70 years | PR-negative |

*This treatment option is under clinical evaluation.

TABLE C
Treatment Options for Women with Axillary
Node-Positive Breast Cancer

| Patient Group | Treatments |
|---|---|
| Premenopausal, | Chemotherapy plus tamoxifen, chemotherapy plus |
| ER(+) or PR(+) | ovarian ablation/GnRH analog, chemotherapy plus |
| | tamoxifen plus ovarian ablation/GnRH analog*, |
| | ovarian ablation alone or with tamoxifen or |
| | GnRH alone or with tamoxifen |
| Premenopausal, | Chemotherapy |
| ER(−) or PR(−) | |
| Postmenopausal, | Tamoxifen plus chemotherapy, tamoxifen alone |
| ER(+) or PR(+) | |
| Postmenopausal, | Chemotherapy |
| ER(−) or PR(−) | |
| Older than 70 | Tamoxifen alone; consider chemotherapy if |
| years | receptor-negative |

*This treatment option is under clinical evaluation.

In the case of epithelial ovarian cancer, aggressive cancer treatment regimens include initial surgical debulking, which includes total abdominal hysterectomy, bilateral salpingo-oophorectomy and omentectomy with cytological evaluation of peritoneal fluid or washings. Adjuvant systemic therapies include combinatorial chemotherapy (e.g. paclitaxel-platinum-based regimens, gecitabine, topotecan, liposomal doxorubicin).

In the case of pancreatic ductal adenocarcinoma, aggressive cancer treatment regimens include surgical resection of the tumor. If the tumor is located in the head of the pancreas, a Whipple operation may be performed to remove the head of the pancreas and part of the small intestine, bile duct and stomach. If the tumor is in the body and tail of the pancreas, a radical distal pancreatectomy is commonly performed, and if the cancer is located in many areas in the pancreas, an extended lymphadenectomy may be needed to remove the entire pancreas, common bile duct, gallbladder, spleen, nearby lymph nodes and portions of the small intestine and stomach. A surgeon may initially perform a laparoscopy to determine if the cancer has spread to other parts of the stomach. Patients are eligible for surgery if there is no detectable metastasis. The majority of patients receive chemotherapy, both adjuvant and neoadjuvant, with some patients also receiving local radiation therapy. Standard front-line chemotherapy is currently gemcitabine. Gemcitabine may be used in combination with other chemotherapeutic agents, including but not limited to, Abraxane (paclitaxel), Folfirinox (combination of 5-fluorouracil, leucovorin, irinotecan and oxaliplatin), fluorouracil, capecitabine, cisplatin, oxaliplatin, and erlotinib.

Examples of drugs that can be used for invasive breast cancer or epithelial ovarian cancer treatment include but are not limited to: bevacizumab, carboplatin, chlorambucil, cisplatin, cetuximab, cyclophosphamide, cytarabine liposomal, docetaxel, epirubicin, erlotinib, fluorouracil, gefitinib, imatinib, mechlorethamine, methotrexate, mitomycin, mitoxantrone, PARP (poly ADP-ribose polymerase) inhibitors, paclitaxel, thiotepa, vinblastine, vinorelbine, trastuzumab, abraxane, doxorubicin, pamidronate disodium, anastrozole, exemestane, raloxifene, toremifene, letrozole, megestrol, tamoxifen, capecitabine, goserelin acetate, and zoledronic acid.

Examples of drugs that can be used for pancreatic ductal adenocarcinoma treatment include but are not limited to: gemcitabine, erlotinib, and taxanes (e.g. docetaxel, paclitaxel), leucovorin, 5-fluorouracil (5-FU), folinic acid, platinum compounds (e.g. oxaliplatin, cisplatin), tyrosine kinase inhibitors (e.g. bevacizumab, cetuximab, erlotinib), doxorubicin, troxacitabine, raltitrexed, pemetrexed, capecitabine, tagafur, topoisomerase-I inhibitors (e.g. irinotecan, camptothecin, topotecan, rubitecan, DX-8951f), proteasome inhibitors (e.g. PS-341), cyclo-oxygenase-2 inhibitors (e.g. celecoxib), 5-lipoxygenase and thromboxane A2 inhibitors (e.g. CV6504), histone deacetylase inhibitors (e.g. CI-994), farnesyl transferase inhibitors (e.g. R115777), mitomycin C, and streptozotocin

### Identification of Tumor Subtypes

In some embodiments, the prognostic and treatment methods further comprise the classification of solid tumor subtype, using methods known in the art.

A. Breast Cancer

Breast cancers are categorized as basal, luminal A, luminal B, or triple-negative subtypes using immunohistochemistry as previously described (Carey et al., JAMA, 295, 2492-2502, 2006). Basal tumors are defined as estrogen receptor (ER) negative, progesterone receptor (PR) negative, HER2 negative and epidermal growth factor receptor (EGFR) positive. Luminal A tumors are defined as ER, PR and HER2 positive. Luminal B tumors are defined as ER and PR positive, and HER2 negative. Triple negative tumors are as ER, PR and HER2 negative. Basal tumors are a subset of triple negative tumors.

B. Ovarian Cancer

The classification of ovarian epithelial tumors currently used by pathologists is based entirely on tumor cell morphology (see e.g., Tavassoli, World Health Organization: Tumours of the Breast and Female Genital Organs, IARC WHO Classification of Tumours, 2003; and Gilks et al., Hum Pathol, 39:1239-1251, 2004). The four major types of epithelial tumors (serous, endometrioid, clear cell, and mucinous) bear strong resemblance to the normal cells lining different organs in the female genital tract. For example, serous, endometrioid, and mucinous tumor cells exhibit morphological features similar to non-neoplastic epithelial cells in the fallopian tube, endometrium, and endocervix, respectively. As approximately 70% of ovarian cancers are serous, ovarian tumors can be categorized as serous or non-serous (Cho et al Annu Rev Pathol, 4:287-313, 2009).

C. Pancreatic Cancer

The classification of pancreatic tumors currently used are based on a variety of techniques including physical examination, histopathology, computed tomography scans, positron emission tomorgraphy scans, endoscopic ultrasound, endoscopic retrograde cholangiopancreatography, percutaneous transhepatic cholangiography, blood tests (e.g. carbohydrate antigen 19.9 levels) and liver function tests (e.g. conjugated bilirubin, γ-glutamyl transpeptidase and alkaline phosphatase levels). The three major types of pancreatic cancers are exocrine pancreatic cancer, pancreatic cystic neoplasms, and pancreatic neuroendocrine tumors. The most common form of exocrine pancreatic cancer is pancreatic ductal adenocarcinoma (PDAC).

### Kits

In another embodiment, a kit of reagents capable of detecting CD4, CD8, and CD68 molecules in a tumor sample is provided. Reagents capable of detecting CD4, CD8 and CD68 molecules include but are not limited to anti-CD4, anti-CD8, and anti-CD68 antibodies, and nucleic acids capable of forming duplexes with CD4, CD8, or CD68 mRNA. Nucleic acids capable of forming duplexes with CD4, CD8 or CD68 mRNA include DNA or RNA sequences, which are complementary to the respective mRNA sequence. Reagents capable of detecting CD4, CD8 and CD68 molecules are also typically directly or indirectly linked to a molecule such as a fluorophore or an enzyme, which can catalyze a detectable reaction to indicate the binding of the reagents to their respective targets.

In another embodiment, a kit of reagents capable of detecting CD8, and CD68 molecules in a tumor sample is provided. Reagents capable of detecting CD8 and CD68 molecules include but are not limited to anti-CD8, and anti-CD68 antibodies, and nucleic acids capable of forming duplexes with CD8, or CD68 mRNA. Nucleic acids capable of forming duplexes with CD8 or CD68 mRNA include DNA or RNA sequences, which are complementary to the respective mRNA sequence. Reagents capable of detecting CD8 and CD68 molecules are also typically directly or indirectly linked to a molecule such as a fluorophore or an enzyme, which can catalyze a detectable reaction to indicate the binding of the reagents to their respective targets.

In some embodiments, the kits further comprise instructions for assessing risk of poor clinical outcome. As used herein, the term “instructions” refers to directions for using the reagents contained in the kit for the detection of the presence of an immune signature of poor outcome or an immune signature of favorable outcome in a sample from a subject. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

### DEFINITIONS

To facilitate an understanding of the embodiments disclosed herein, a number of terms and phrases are defined below.

The phrase “increased risk of poor clinical outcome” when used herein in relation to detection of an immune signature indicates that a human patient has a greater likelihood of having a poor clinical outcome when an immune signature of poor outcome is detected than when said immune signature of poor outcome is not detected. Numerically an increased risk is associated with a hazard ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for overall survival or recurrence-free survival.

Aggressive treatment regimen: A cancer treatment regimen in which the emphasis is on killing and/or removing the cancer from the body as thoroughly as possible, to the possible detriment of patient comfort and/or safety. As compared to a conservative treatment regimen, an aggressive treatment regimen may, for example, use higher doses of anti-cancer therapeutics, higher total treatment times, and more radical surgeries.

Conservative treatment regimen: A cancer treatment regimen in which efforts are made to kill and/or remove the cancer from the body, but which also heavily takes into account patient comfort and/or safety. As compared to an aggressive treatment regimen, a conservative treatment regimen may use lower doses of anti-cancer therapeutics, lower total treatment times, and/or less radical surgeries.

Immuno-based/Antibody-based: Any technique that involves the use of an antibody to detect an antigen. Immuno-based techniques include immunostaining, ELISA, antibody microarray, flow cytometry, and western blotting.

Nucleic acid-based: Any technique that involves the use of a nucleic acid to detect another nucleic acid. Nucleic acid includes both DNA and RNA. Nucleic acid-based techniques include nucleic acid microarray, RT-PCR, northern blotting, nuclease protection assays, and in situ hybridization.

### EXPERIMENTAL

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); V (volts); MW (molecular weight); sec (seconds); min(s) (minute/minutes); h(s) and hr(s) (hour/hours); ° C. (degrees Centigrade); QS (quantity sufficient); ND (not done); NA (not applicable); rpm (revolutions per minute); H₂O (water); dH₂O (deionized water); aa (amino acid); by (base pair); kb (kilobase pair); kD (kilodaltons); cDNA (copy or complementary DNA); DNA (deoxyribonucleic acid); ssDNA (single stranded DNA); dsDNA (double stranded DNA); dNTP (deoxyribonucleotide triphosphate); RNA (ribonucleic acid); OD (optical density); PCR (polymerase chain reaction); RT-PCR (reverse transcription PCR).

Additional abbreviations include: CTL (cytotoxic T lymphocyte); Th (helper T lymphocyte); NK (natural killer cell); EOC (epithelial ovarian cancer); ER (estrogen receptor); PR (progesterone receptor); OS (overall survival); pCR (pathologic complete remission); PDAC (pancreatic ductal adenocarcinoma); RFS (recurrence-free survival); IHC (immunohistochemistry); and TMA (tissue microarrays).

### Example 1

### Antibody-Based Method for Determining the Immune Signature of Tumor-Infiltrating Leukocytes in Breast Cancer Patients

This example describes methods for immunophenotyping tumor-infiltrating leukocytes, and provides results of immune signature analyses in two cohorts of breast cancer patients.

Materials and Methods

Patients and Tumor Samples.

Tissue microarray studies were conducted on two separate patient cohorts. The screening cohort, described elsewhere in detail (Brennan et al., Clin Cancer Res, 14:2681-2689, 2008), was constructed from 179 cases of invasive breast cancer diagnosed at the Department of Pathology, Malmo University Hospital, Malmo, Sweden, between 2001 and 2002. The median age at diagnosis was 65 and the median follow-up time for overall survival was 52 months. Patients did not receive neo-adjuvant treatment and were treated with either modified radical mastectomy or wide local excision. The median tumor size was 2.2 cm and 62% of the tumors were PR positive and 72% were ER positive. The second (validation) cohort includes 498 patients with primary invasive breast cancer diagnosed at the Malmo University Hospital between 1988 and 1992 These cases belonged to an original cohort of 512 patients as previously described in detail (Paulsson et al., Am J Pathol, 175:334-341, 2009; and Borgquist et al., J Clin Pathol, 61:197-203, 2008). The median age at diagnosis was 65 years and median follow-up time to first breast cancer event was 128 months. Information regarding the date of death was obtained from the regional cause-of-death registries for all patients in both cohorts. Complete treatment data was available for 379 patients, 160 of whom had received adjuvant tamoxifen. Information on adjuvant chemotherapy was available for 382 patients, of which 23 patients received treatment. 200 patients received no adjuvant treatment.

Immunohistochemistry.

Tissue microarray slide sections (4.0 μm) were deparaffinized in xylene, and re-hydrated through descending concentrations of ethanol. For CD4 and CD8 detection, heat-mediated antigen retrieval was performed using microwave treatment for 7 min in a citrate buffer (BioGenex) followed by 3 serial 5 minute washes in phosphate buffered saline (PBS). Antigen retrieval for CD68 detection was accomplished by treatment of slides with Protinase XXV (Lab Vision Inc.) for 5 minutes followed by 4 serial 5 minute washes in PBS. Endogenous peroxidase activity was blocked by incubating slides in 5% hydrogen peroxide (Sigma) diluted in methanol for 20 min, followed by 4 serial washes in PBS. To reduce nonspecific background, slides were pretreated with blocking buffer (10% goat serum (Invitrogen), 1.0% bovine serum albumen (Sigma) dissolved in PBS for 30 min. Primary antibodies were pre-diluted in blocking buffer to 1:100 for CD68 (KP-1, NeoMarkers) and CD8 (C8/144B, NeoMarkers) or 1:25 for CD4 (368, Novocastra) and applied to tissue section for 16 hours at 4° C. Signal amplification and development were accomplished utilizing the Ultravision LP Detection System (Thermo Scientific) according to manufactures guidelines. Additional CD4 antibodies were tested including clone 34930 (R&D systems) and clone 4B.12 (Noemarkers) but yielded sub-optimum staining and thus were not used for analysis.

Automated Image Acquisition, Management, and Analysis.

Fully automated image acquisition was employed herein. The Aperio ScanScope XT Slide Scanner (Aperio Technologies, Vista, Calif.) system was used to capture whole slide digital images with a 20× objective. Slides were de-arrayed to visualize individual cores, using Spectrum software (Aperio). A tumor nuclear algorithm (IHCMark) was developed in house to quantify DAB positive immune cells (Rexhepaj et al., Breast Cancer Res, 10:R89, 2008). The algorithm calculated the density of immune cells/mm².

Manual and Semi-Automated Evaluation of Immunohistochemical Staining.

As an alternative to fully automated signal assessment, semi-automated and manual assessment of leukocyte infiltration in tumor specimens is possible. Briefly, CD68, CD8 and CD4 positive cells are scored at 20× by two independent pathologists and averaged for continuity. In studies using TMAs leukocyte density is quantitated by counting all high power fields (20×) per tissue section (1.1 mm)/2 sections/patient. In studies using human whole tissue samples, 10 high power fields (20×) are accessed and averaged to generate a leukocyte density score. This can be done completely manually, using a bright light microscope. Alternatively leukocyte infiltration can be assessed by semi-automated image capture at 10× magnification by OpenLab (Improvision/PerkinElmer) and quantitation of positive cells utilizing ImageJ (NIH). Briefly, image capture is accomplished on standard Leica DC500 microscope equipped with digital camera. Images are then exported as tiff files and loaded into ImageJ (NIH). ImageJ software is utilized to record manual quantitation and allows for samples to be processed in bulk. For these manually quantitated data, leukocyte infiltration characterized as “high” using a 75th percentile cut-off from the mean leukocyte infiltration for the assessed sample population. All other samples are deemed to fall in the “low” infiltration group.

Image and Statistical Analysis.

Image analysis and statistical modeling was accomplished using methods described in detail elsewhere (Brennan et al., Clin Cancer Res, 14:2681-2689, 2008; and Rexhepaj et al., Breast Cancer Res, 10:R89, 2008). Briefly, the leukocyte infiltration was scored as the total number of leukocytes expressing the marker of interest (e.g., CD68, CD8, CD4) over the total number of tumor cells. The final value of leukocyte infiltration quantitated for each patient was the maximum out of both tissue cores. Patients with less than 200 cells in both tissue cores were discarded from analysis. A classification tree procedure was used to create a tree-based classification model and cases were classified into groups based on a the values of dependent predictor variable (target). Patient survival was used as the target variable for building the predictions trees. These tree models were evaluated in terms prediction accuracy using a 10-fold cross-validation approach. The decision tree with the highest accuracy was selected as optimal for the dataset. Kaplan-Meier analysis and the log-rank test were used to illustrate differences between overall survival (OS) according to individual CD68, CD4, and CD8 expression. A Cox regression proportional hazards model was employed to estimate the relationship to OS of the CD68/CD4/CD8 immune profile, lymph node status, tumor grade, and HER2, PR and ER status in the patient cohorts. Multivariate models included any variable that displayed a significant association with outcome following univariate analysis. A p-value of <0.05 was considered statistically significant and all calculations were performed using Statistical Package for the Social Sciences (SPSS, Inc.). Random forest clustering (RFC) was performed using R software.

Results

Single Immune Marker Analysis of Leukocyte Infiltrates.

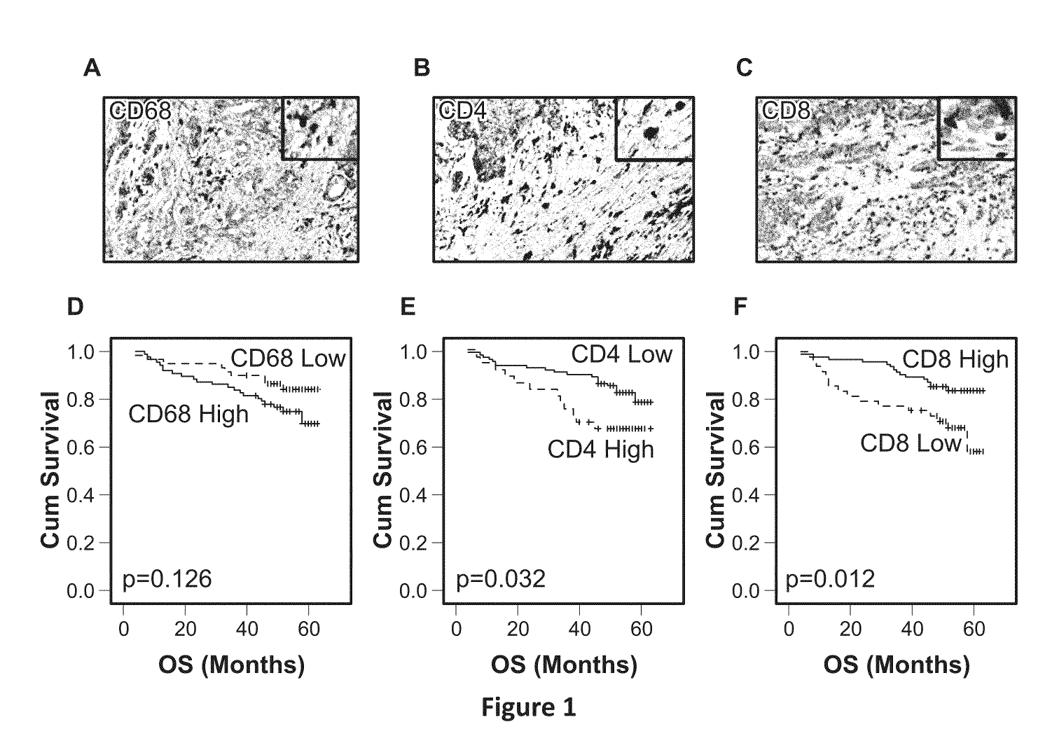

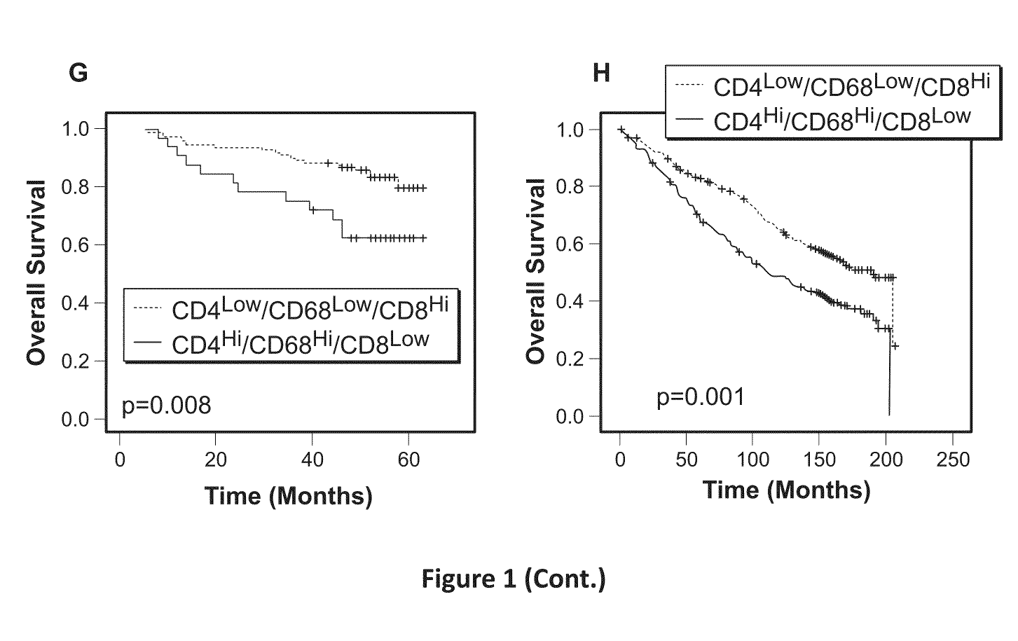

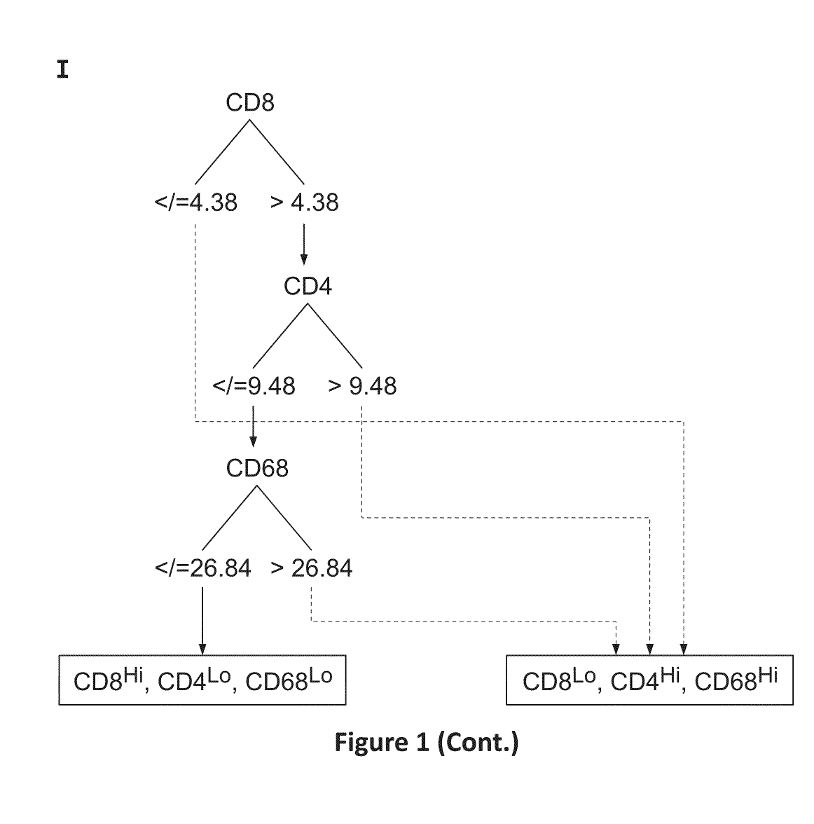

Previous work utilizing transgenic mouse models of mammary carcinogenesis has revealed a tumor-promoting role for TH2-CD4+ T lymphocytes that elicit pro-tumor, as opposed to cytotoxic bioactivities of tumor-associated macrophages and enhancement of pro-metastatic epidermal growth factor receptor signaling programs in malignant mammary epithelial cells (DeNardo et al., Cancer Cell, 16:91-102, 2009). Without being bound by theory, in addition to the underlying genetic alterations in malignant epithelial cells, the host immune response to neoplasia in the breast was determined during development of the present disclosure to contribute to the histopathologic features of breast cancer. CD4⁺, CD8⁺ and CD68⁺ leukocyte density was assessed by immunohistochemistry using a tissue microarray (TMA) consisting of tumor tissue representing two independent cohorts of breast cancers (FIG. 1A-F). Following digital scanning of stained TMA slides using an Aperio ScanScope XT slide scanner, a fully automated nuclear algorithm was used to discriminate tumor from “normal” tissue, and to quantify CD4⁺, CD8⁺ and CD68⁺ cells. Random forest clustering was employed to identify optimum thresholds for survival analysis. Kaplan Myer analysis for overall survival demonstrates that as single variables “high” infiltration by CD4⁺ cells and “low” CD8⁺ cell density predict reduced overall survival, while CD68⁺ cell density alone showed no statistical difference in overall survival (FIG. 1D-F).

Three-Marker Profile of Leukocyte Infiltrates.

Heterotypic interactions between diverse leukocytes populations often determine the outcome of an immune response. Thus combining the predictive power of CD4, CD8 and CD68 should significantly stratify patients represented on the TMA for overall survival by accessing both anti-tumor immune response (e.g., CD8⁺ density), as well as pro-tumor immune responses capable of promoting metastatic spread (e.g., high CD4⁺ and CD68⁺ density). Accordingly, an immune profile of CD68^{lo}/CD4^{lo}/CD8^{hi} was predicted to represent individuals whose breast tumors were controlled by local resection of primary tumor and adjuvant therapy (e.g., patients exhibiting longer relapse-free survival). In contrast, patients bearing an immune response characterized as CD68^{hi}/CD4^{hi}/CD8^{lo} were predicted to represent a population of patients at risk for metastasis, relapse and reduced overall survival. Random forest clustering was employed to identify optimum thresholds for discriminating CD68^{hi}/CD4^{hi}/CD8^{lo} and CD68^{lo}/CD4^{lo}/CD8^{hi} in survival analysis using patient cohort I (FIG. 1I). Utilizing thresholds set for discriminating CD68^{hi}/CD4^{hi}/CD8^{lo} and CD68^{lo}/CD4^{lo}/CD8^{hi} from cohort I this signature was verified in a second independent cohort II including 498 samples with a median follow-up time of 128 months. Kaplan Meier analysis for these two groups demonstrated significantly reduced overall survival (OS) in patients bearing the CD68^{hi}/CD4^{hi}/CD8^{lo} IHC signature (p=0.008, p=0.001; FIG. 1G-H). In addition, Log Rank (Mantel-Cox) analysis revealed the CD68^{hi}/CD4^{hi}/CD8^{lo} signature (3 marker signature) had an increased survival hazard ratio (HR), in cohorts one and two as shown in Table 1-1, and Tables 1-2a (initial calculations) and 1-2b (final calculations including additional clinical data). The CD68^{hi}/CD4^{hi}/CD8^{lo} signature was also associated with decreased recurrence-free survival (RFS) in both cohorts (Table 1-3 and Table 1-4). Multivariate Cox regression analysis revealed that the CD68^{hi}/CD4^{hi}/CD8^{lo} signature was an independent predictor of decreased OS and RFS after controlling for disease grade, nodal status, tumor size, ER expression, PR expression, HER2 positively and Ki67 status in both cohorts, indicating that the immune signature predicts breast cancer survival independently of the typical clinical histopathological markers currently employed.

TABLE 1-1
Cox Regression Analysis of Overall
Survial - Breast Cancer Cohort One

95% CI

| | HR | Lower | Upper | P Value |
|---|---|---|---|---|
| Grade | 0.556 | 0.186 | 1.665 | 0.294 |
| Nodal Status | 2.461 | 0.939 | 6.449 | 0.067 |
| Size | 1.152 | 0.400 | 3.316 | 0.793 |
| ER | 0.535 | 0.137 | 2.090 | 0.368 |
| PR | 0.541 | 0.195 | 1.501 | 0.238 |
| Age | 1.033 | 0.999 | 1.069 | 0.057 |
| Her2 | 1.430 | 0.971 | 2.106 | 0.070 |
| Ki67 | 8.388 | 1.693 | 41.561 | 0.009 |
| 3 marker sig. | 3.510 | 1.342 | 9.18 | 0.01 |

TABLE 1-2a
Cox Regression Analysis of Overall
Survival - Breast Cancer Cohort Two

95% CI

| | HR | Lower | Upper | P Value |
|---|---|---|---|---|
| Grade | 1.892 | 1.331 | 2.690 | <0.001 |
| Nodal Status | 2.613 | 1.900 | 3.593 | <0.001 |
| Size | 1.003 | 0.996 | 1.011 | 0.371 |
| ER | 0.623 | 0.374 | 1.039 | 0.070 |
| PR | 0.971 | 0.619 | 1.523 | 0.899 |
| Age | 1.052 | 1.038 | 1.066 | <0.001 |
| Her2 | 1.003 | 0.597 | 1.685 | 0.991 |
| Ki67 | 0.967 | 0.680 | 1.375 | 0.852 |
| 3 marker sig. | 1.377 | 1.016 | 1.865 | 0.039 |

TABLE 1-2b
Cox Regression Analysis of Overall
Survival - Breast Cancer Cohort Two

95% CI

| | HR | Lower | Upper | P Value |
|---|---|---|---|---|
| Grade | 1.900 | 1.241 | 2.909 | 0.003 |
| Nodal Status | 2.394 | 1.627 | 3.524 | <0.001 |
| Size | 1.006 | 0.997 | 1.014 | 0.178 |
| ER | 0.836 | 0.483 | 1.446 | 0.522 |
| PR | 0.495 | 0.321 | 0.763 | 0.001 |
| Age | 1.047 | 1.029 | 1.065 | <0.001 |
| Her2 | 1.128 | 0.806 | 1.241 | 0.999 |
| Ki67 | 0.867 | 0.558 | 1.347 | 0.527 |
| 3 marker sig. | 1.676 | 1.142 | 2.460 | 0.008 |

TABLE 1-3
Cox Regression Analysis of Recurrence Free
Survival - Breast Cancer Cohort One

95% CI

| | HR | Lower | Upper | P Value |
|---|---|---|---|---|
| Grade | 1.266 | 0.412 | 3.888 | 0.681 |
| Nodal Status | 5.081 | 1.726 | 14.95 | 0.003 |
| Size | 1.038 | 1.016 | 1.060 | 0.001 |
| ER | 0.264 | 0.063 | 1.107 | 0.069 |
| PR | 1.349 | 0.389 | 4.676 | 0.637 |
| Age | 1.033 | 0.997 | 1.070 | 0.072 |
| Her2 | 6.170 | 2.015 | 18.89 | 0.001 |
| Ki67 | 0.262 | 0.029 | 2.374 | 0.233 |
| 3 marker sig. | 2.788 | 1.002 | 7.772 | 0.050 |

TABLE 1-4
Cox Regression Analysis of Recurrence Free
Survival - Breast Cancer Cohort Two

95% CI

| | HR | Lower | Upper | P Value |
|---|---|---|---|---|
| Grade | 2.024 | 1.181 | 3.468 | 0.010 |
| Nodal Status | 2.724 | 1.643 | 4.514 | <0.001 |
| Size | 0.998 | 0.998 | 1.009 | 0.748 |
| ER | 1.523 | 0.807 | 2.873 | 0.194 |
| PR | 0.327 | 0.188 | 0.570 | <0.001 |
| Age | 0.998 | 0.980 | 1.015 | 0.784 |
| Her2 | 0.965 | 0.747 | 1.246 | 0.785 |
| Ki67 | 1.441 | 0.787 | 2.638 | 0.237 |
| 3 marker sig. | 2.368 | 1.433 | 3.912 | 0.001 |

Immune Signature Predicts Breast Cancer Survival Independently of Tumor Subtype.

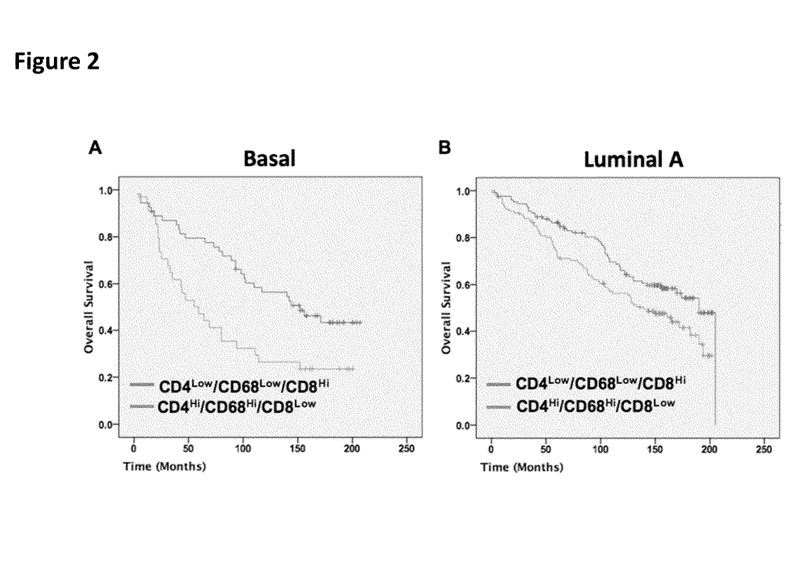

The CD68/CD4/CD8 signature was further studied to determine if it correlated with an individual tumor subtype (such as basal or luminal, etc). Multivariate Cox regression analysis of tumor subtypes and the CD68/CD4/CD8 signature demonstrated that indeed the three-marker based signature was independent of luminal B, HER2-positive, basal type or even un-typed triple negative (ERα, PR, HER2 negative) tumors. The CD68/CD4/CD8 signature significantly predicted survival in luminal A and basal tumors, but not luminal B and HER2 positive tumors (FIG. 2A-B and Table 1-5). These analyses indicate that the three-marker immune-based signature is a useful predictor of overall survival for multiple breast cancer subtypes, and as such is an improvement to existing gene expression-based prognostic profiling methods to evaluate risk. In particular this immune signature is predictive for patients with low-risk ER⁺ tumors (such as in the luminal A subtype) or alternatively high-risk triple negative breast tumors.

TABLE 1-5
Log-Rank (Mantel-Cox) Analysis For The CD68/CD4/CD8
Signature in Various Breast Tumor Subtypes

| Molecular Subtype | Chi-Square | dF | Sig. |
|---|---|---|---|
| Basal | 7.405 | 1 | 0.007 |
| Her2+/ER− | 0.009 | 1 | 0.922 |
| Luminal A | 5.486 | 1 | 0.19 |
| Luminal B | 1.024 | 1 | 0.312 |

Tissue microarrays containing specimens representing various grades of ductal carcinoma in situ (DCIS) are also prepared and examined. The three marker immune-based signature is also expected to stratify patients with this common noninvasive type of breast cancer.

Three-Marker Immune Signature is an Independent Predictor of Recurrence Free Survival (RFS) in Node-Positive Patients.

The overall survival (OS) of breast cancer patients is greatly reduced if metastasis to regional or draining lymph nodes is present at the time of primary tumor detection. Therefore, node-positive patients require aggressive treatment with neoadjuvant or adjuvant systemic chemotherapy, or targeted therapies such as anti-estrogens or trastuzumab. To assess whether immune infiltration by macrophages and T lymphocytes affected the survival of this high-risk group, the impact of the CD68/CD4/CD8 signature was examined following stratification for nodal status. Whereas the CD68/CD4/CD8 signature was not predictive in node-negative patients, Kaplan-Meier analysis of cohort II demonstrated significantly reduced RFS in node-positive patients whose tumors harbored the CD68^{hi}/CD4^{hi}/CD8^{lo} signature. Multivariate Cox regression analysis revealed that the CD68^{hi}/CD4^{hi}/CD8^{lo} signature was an independent predictor of decreased RFS after controlling for grade, tumor size, ER, PR, HER2, and ki67 status. Thus, tumor infiltration by macrophages and T lymphocytes may influence breast cancer recurrence in lymph node-positive patients, a group often aggressively treated with neoadjuvant and adjuvant chemotherapy.

### Example 2

### Nucleic Acid-Based Method for Determining the Immune Signature of Tumor-Infiltrating Leukocytes in Cancer Patients

This example describes methods for assessing mRNA levels of three biomarkers of tumor-infiltrating leukocytes, and for retrospectively analyzing the immune signature in published gene expression data sets.

Assessing mRNA of Biomarkers of Tumor-Infiltrating Leukocytes.

Isolation and measurement of mRNA from tumor samples can be performed as known in the art (Micke et. al., Laboratory Investigation, 86, 202-211, 2006). Tumor samples are removed from a patient and frozen in liquid nitrogen. The tumor samples are thawed, and cut into approximately 20 micrometer-thick sections. Each section of tumor is placed in a tube containing 300 microliters Trizol (Invitrogen). Sixty microliters of chloroform-isoamylalcohol is then added to the Trizol-tumor sample mixture. The tube is mixed, and centrifuged at 10,000 rpm for 10 minutes. Two liquid phases form during the centrifugation, and after the end of the spin, the aqueous phase is transferred to a new tube and 2 microliters of co-precipitant (PelletPaint, Novagen) and 1 equivalent volume (approximately 160 microliters) isopropanol is added. The tube is mixed and incubated at room temperature for 5 minutes, and then centrifuged at 13,000 rpm for 10 minutes at room temperature, which precipitates the RNA. The supernatant is poured off, and the RNA pellet is then washed twice with 70% ethanol. The 70% ethanol is poured off, and the RNA pellet is dried. The RNA pellet is then resuspended in 20 microliters RNAse-free water.

Next, the re-suspended RNA is used a template for cDNA synthesis. One microliter of oligo-dT primers (dT17) and 1 microliter 10 nM dNTPmix (Clonetech) is added to 5 microliters of each resuspended RNA solution. The mixtures are heated for 10 minutes at 65 degrees Celsius, and then immediately chilled on ice. To each tube, 4 microliters of first strand buffer, 2 microliters DTT, 1 microliter RNAsin (Promega) and 1 microliter Superscript II reverse transcriptase (Invitrogen) are added, and then the reactions are kept at 42 degrees Celsius for 1 hour to allow reverse transcription to occur. After 1 hour, the reaction is stopped by moving the tubes to 65 degrees Celsius for 20 minutes. Next, the RNA is digested with 1 microliter RNAseH (Clonetech) at 37 degrees Celsius for 30 minutes.

For the PCR, 2 microliters of the cDNA solution (10%) is used for each 40-cycle Sybgreen PCR assay using the Sybrgreen Universal PCR Master Mix (Applied Biosystems) with forward and backward primers for the CD4, CD8, or CD68 cDNA. The PCR reaction is performed with an ABI PRISM 7000HT real-time PCR cycler (Applied Biosystems) using conditions recommended by the manufacturer. Gene expression levels are normalized to expression of the housekeeping gene glyceraldehyde-3-phosphate dehydrogenase (GAPDH).

A profile of a combination of high CD68, high CD4, and low CD8 mRNA levels is contemplated to be associated with a greater risk of tumor relapse and/or metastasis as compared to a profile of a combination of low CD68, low CD4, and high CD8 mRNA levels.

Validation of Immune Profile in Existing Expression Data Bases.

Data base mining is done to assess the predictive power of the CD68/CD4/CD8 signature in published data sets. Assessments of single gene expression changes have demonstrated that both CD68 and CD4 genes are enriched in tissue from patients who have relapsed within 5 years, while CD8 is down regulated in tissue of patients who have been in remission during the 5-year follow up time period. The CD68/CD4/CD8 gene expression prognostic signature is assessed by multi-variant analysis using random forest clustering to evaluate cut off points, and the signature is used to improve risk stratification independently of known clinicopathologic factors and previously established prognostic signatures based on unsupervised hierarchical clustering (“molecular subtypes”) or supervised predictors of metastasis (“multi-gene prognosis signature”).

### Example 3

### Antibody-Based Method for Determining the Immune Signature of Tumor-Infiltrating Leukocytes in Ovarian Cancer Patients

This example describes methods for immunophenotyping tumor-infiltrating leukocytes, and provides results of immune signature analyses in a cohort of epithelial ovarian cancer (EOC) patients.

Materials and Methods

Patients and Tumor Samples.

The tissue microarray (TMA) used in this study, described elsewhere in detail (Brennan et al., Eur J Cancer, 45:1510-1517, 2009), was constructed from a consecutive cohort of 76 patients diagnosed with primary invasive epithelial ovarian cancer at the National Maternity Hospital, Dublin, with a median follow-up of 4.3 years. The standard surgical management was a total abdominal hysterectomy, bilateral salpingo-oophorectomy and omentectomy with cytological evaluation of peritoneal fluid or washings. Residual disease was resected to less than 2 cm where possible. Stage and volume of residual disease (no residual disease, residual disease greater or less than 2 cm) were recorded in all cases. All patients received adjuvant chemotherapy consisting of cisplatin or carboplatin prior to 1992 and combined with paclitaxel from 1992 to 2002. No patient received neo-adjuvant chemotherapy. Benign or borderline ovarian cancers, non-epithelial ovarian cancer and cases with histological features typical of secondary ovarian cancer were excluded from the study. Diagnostic specimens were all formalin fixed and paraffin embedded in the Department of Pathology at the National Maternity Hospital, Dublin, Ireland. All tissue blocks were stored in that department prior to construction of the tissue microarray. Full ethical approval was obtained from the Ethics Committee of the National Maternity Hospital, Dublin and informed consent was obtained from living patients and relatives of deceased patients.

Tissue Microarrays.

Seventy six paraffin-embedded tumor specimens were used for tissue microarray construction as previously described (Brennan et al., 2009, supra). Areas representative of invasive cancer were marked on haematoxylin and eosin-stained slides and the tissue microarray was constructed using a manual tissue arrayer (MTA-1, Beecher Inc, Wis.). The array consisted of four cores per patient. Two 1.0 mm cores were extracted from each donor block and assembled in a recipient block. Recipient blocks were limited to approximately 100 cores each. In general, cores were taken from the peripheral part of the tumor in cases where the tumor had well-defined borders. In more diffusely growing tumors, areas with the highest tumor cell density were primarily targeted. Necrotic tissue was avoided.

Immunohistochemistry.

Tissue microarray slide sections were prepared and immunohistochemistry was performed as described in Example 1.

Image acquisition and analysis.

The Aperio ScanScope XT Slide Scanner (Aperio Technologies) system with a 20× objective was used to capture whole-slide digital images. Slides were de-arrayed to visualize individual cores, using Spectrum software (Aperio). A tumor nuclear algorithm (IHCMark) was developed in-house to quantify the density of DAB positive immune cells/mm² (Rexhepaj et al., Breast Cancer Res., 10:R89, 2008).

Statistics.

Decision tree analysis was performed on the tissue microarray data. For this purpose, all patients were randomly divided in 10 subsets. A decision tree model was selected using a 10-fold cross-validation approach. Ten consecutive decision tree models were independently constructed using the CD4, CD8 and CD68 continuous output from 9 subsets. The prognostic accuracy of each decision tree model was tested using the remaining set of patients, with the model displaying the highest accuracy being selected as the optimal model for the dataset. Kaplan-Meier analysis and the log-rank test were used to illustrate differences between recurrence free survival according to individual CD68, CD4, and CD8 expression. A Cox regression proportional hazards models was employed to estimate the relationship between recurrence free survival and the CD68/CD4/CD8 immune profile, disease stage, tumor grade, age at diagnosis and residual disease after primary surgery. A P value of <0.05 was considered statistically significant and all calculations were performed using SPSS version 12.0 (SPSS Inc, Chicago, Ill.).

Validation of Immune Signature in a Second Large Cohort.

A validation cohort of 154 ovarian cancer cases collected from two prospective, population-based cohorts, the Malmo Diet and Cancer Study (MDCS) and Malmo Preventive Project (MPP) have now been screened. The MDCS was initiated in 1991 and enrolled 17,035 healthy women (Berglund et al., J Intern Med, 223:45-51, 1993). The MPP was established in 1974 for screening with regard to cardiovascular risk factors and enrolled 10,902 women (Berglund et al., J Intern Med, 239:489-497, 1996). Median follow-up for this cohort is 2.67 years (range 0-21.1 years) at which point 105 patients were dead, 98 from ovarian cancer. Results from these studies are expected to extend the predictive power of the CD68/CD4/CD8 signature to different histological subtypes.

### Results

Single Immune Marker Analysis of Leukocyte Infiltrates.

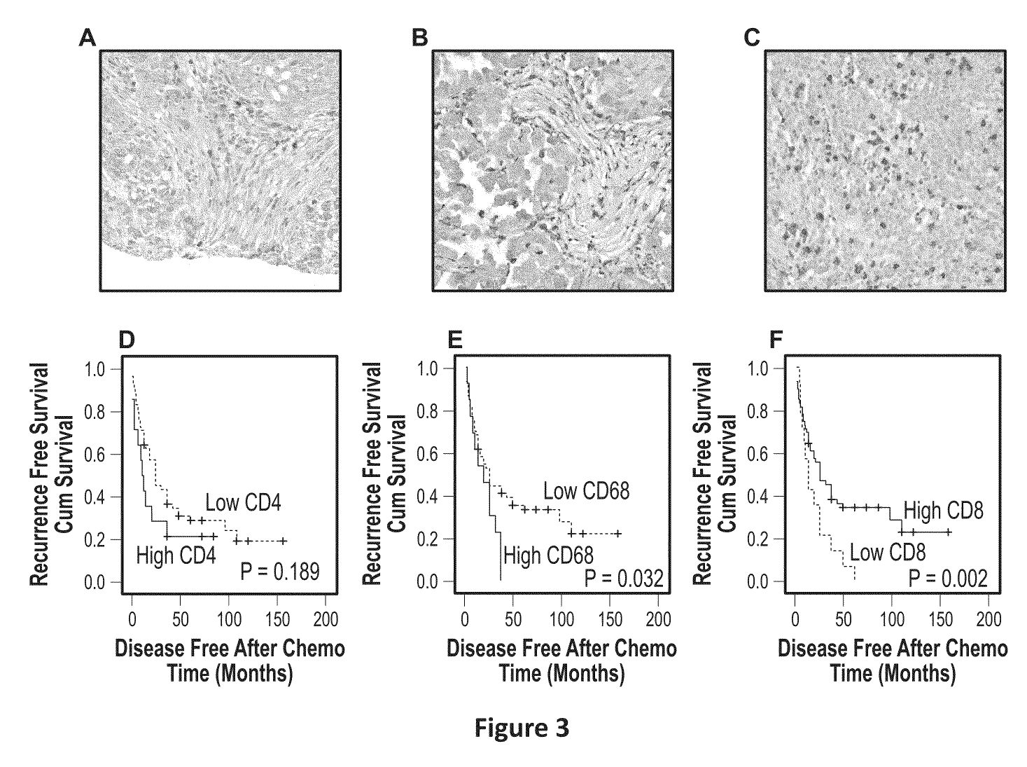

After finding a correlation between the survival of breast cancer patients and the CD68/CD4/CD8 signature of their tumor infiltrating leukocytes (Example 1), the applicability of this signature to epithelial ovarian cancer was assessed. First, individual CD4⁺, CD8⁺ and CD68⁺ leukocyte densities were analyzed by immunohistochemistry using a tissue microarray (TMA) consisting of tumor tissue representing 76 epithelial ovarian cancers (FIG. 3). After digital scanning of stained TMA slides using an Aperio ScanScope XT slide scanner, a fully automated nuclear algorithm was employed to quantify CD4⁺, CD8⁺ and CD68⁺ cells. For survival analysis, high and low thresholds for each marker were established by decision tree analysis with 10-fold cross-validation of tree models. Finally, Kaplan Myer analysis of recurrence free survival (RFS) demonstrated that as single variables high infiltration by CD68⁺ cells and low CD8⁺ cell density predict reduced RFS, while CD4⁺ cell density alone showed no statistical difference in overall survival (FIG. 3D-E).

Three-Marker Profile of Leukocyte Infiltrates.

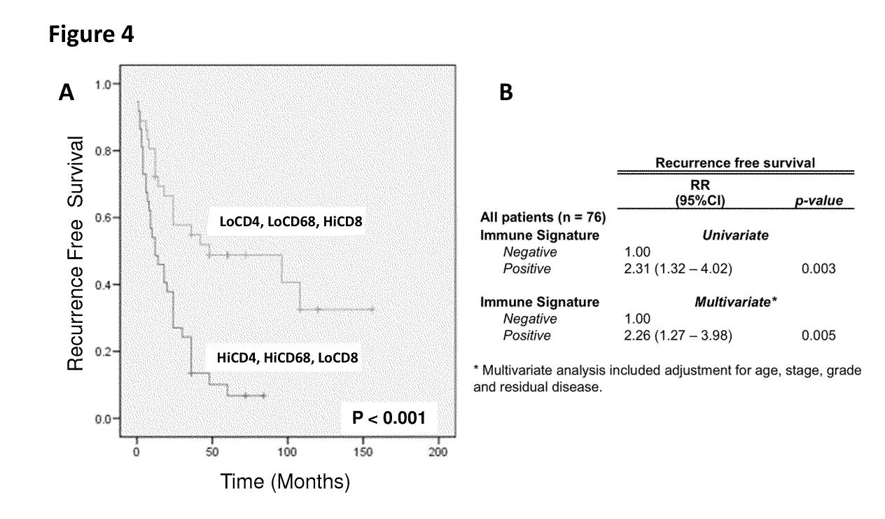

Next, the predictive power of CD4, CD8, and CD68 was combined to stratify EOC patients for RFS. A classification and regression trees algorithm was used to define the signature. High and low thresholds for each marker were established through decision tree analysis with 10-fold cross-validation of tree models. All patients were categorized as having 1) CD68^{hi}/CD4^{hi}/CD8^{lo} or 2) CD68^{lo}/CD4^{lo}/CD8^{hi}. Kaplan Myer analysis of these two groups demonstrated significantly reduced RFS in patients bearing the CD68^{hi}/CD4^{hi}/CD8^{lo} immunohistochemistry signature (p<0.001; FIG. 4A). In addition, multivariate Cox regression analysis revealed the CD68/CD4/CD8 signature had an increased survival hazard ratio of 2.26 and no statistical correlation with disease grade, stage, age at diagnosis or post-operative residual disease (FIG. 4B), indicating that the immune signature predicts epithelial ovarian cancer survival independently of the typical clinical and histopathological markers currently employed.

Immune Signature Predicts Epithelial Ovarian Cancer Survival in Serous Subtype.

Epithelial ovarian cancer is known to be a heterogeneous disease, the entities of which are in part reflected in traditional histopathological characteristics. Therefore, biomarkers were assessed separately in histological subgroups, as well as across the entire patient cohort. Consequently, it is critical for the utility of the CD68/CD4/CD8 signature to predict the outcome in individual tumor histological subtypes of ovarian cancer (e.g., serous versus non-serous).

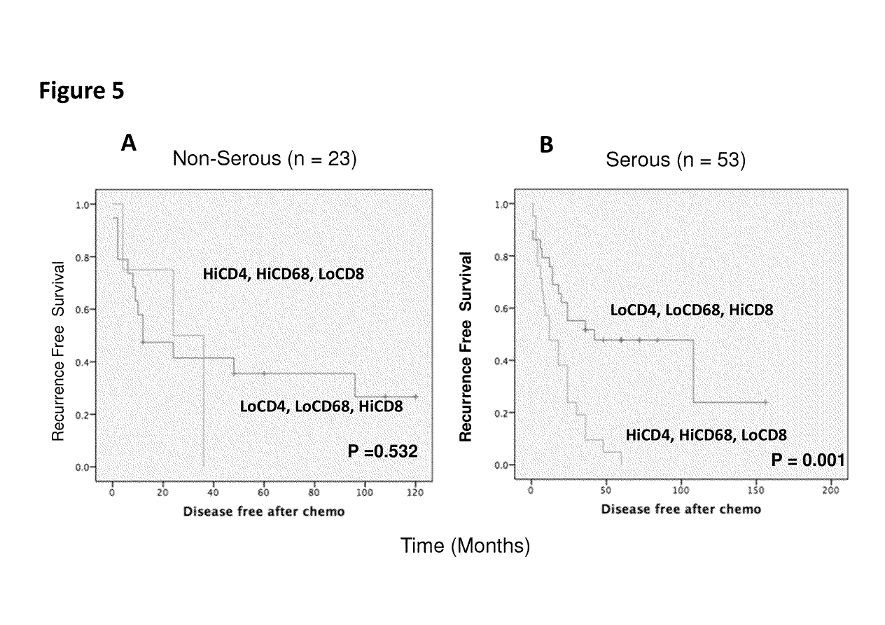

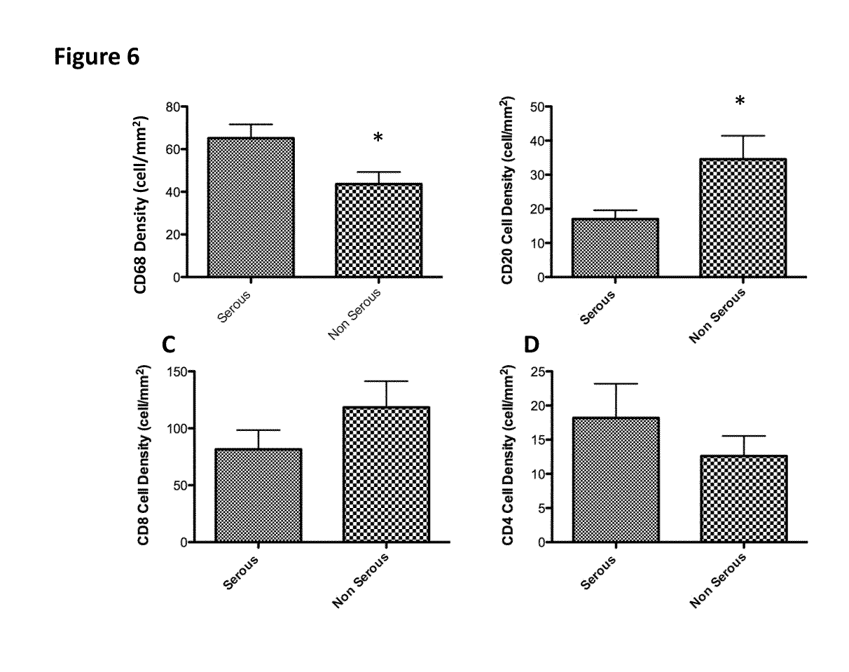

Kaplan Meier analysis demonstrated that the CD68/CD4/CD8 signature was associated with a reduced RFS in serous (n=53) tumors (p=0.001) (FIG. 5B), but not non-serous tumors (n=23) (FIG. 5A). Analysis of the leukocyte infiltrate in serous compared to non-serous tumors further revealed that the mean CD68 cell density (p=0.037) was higher and the mean CD20 cell density (p=0.004) was lower in serous tumors. A non-significant trend towards a lower CD8 and higher CD4 cell density in serous tumors was also detected (FIG. 6).

### Example 4

### Nucleic Acid-Based Analysis Method for Predicting Patient Disease Recovery and Response to Chemotherapy

This example describes methods for assessing mRNA levels of two biomarkers of tumor-infiltrating leukocytes from previously published gene expression data sets and indicates that stratification of biomarker expression levels is predictive of both disease recovery and response the chemotherapy.

### Materials and Methods

Retrospective Gene Expression Survival Analysis.

Gene expression data sets were downloaded from the Gene Expression Omnibus (“www.ncbi.nlm.nih.gov/geo”) or authors' websites in the form of raw data files where possible. Only datasets with clinical survival information and at least 50 patients were included. Table 4-1 provides a list of the datasets used. In total 3,872 samples across 14 different platforms were analyzed. Where raw data was not available, the normalized data as published by the original study was used. In the case of the Affymetrix datasets (.cel files), gene expression values were called using the robust multichip average method (Irizarry et al., Biostatistics, 4: 249-264, 2003) and data were quantile normalized using the Bioconductor package, affy (“www.bioconductor.org”). For the dual-channel platforms, data were loess normalized (Yang et al., In Microarrays: Optical Technologies and Informatics, eds. Bittner et al., Proceedings of SPIE, pp. 141-152, 2001) using the Bioconductor package limma. Hybridization probes were mapped to Entrez gene IDs (Maglott et al., Nucleic Acids Res 35, D26-31, 2007). The Entrez gene IDs corresponding to the array probes were obtained using Biomart (“www.biomart.org”) and the Bioconductor annotation libraries. Probes that hit multiple genes were filtered out. If there were multiple probes for the same gene, the probes were averaged for that gene. All calculations were carried out in the R statistical environment (“cran.r-project.org”).

Relapse-free survival (RFS) of untreated patients was considered the survival end point. When RFS was not available, distant metastasis-free survival (DMFS) data was used, and if neither RFS nor DMFS were available, then overall survival (OS) was used. Median expression for both CD8 and CD68 was used to determine high and low groups within each of the 22 individual datasets. Once a sample was assigned to a particular group the 22 datasets were combined and a global survival analysis was performed. It is important to treat each dataset separately when determining if a sample belongs to the high or low expression groups, as the expression of the CD8 and CD68 will vary greatly across the different experiments or platforms. The survival curve was based on Kaplan-Meier estimates. The R package survival was used to calculate and plot the Kaplan-Meier survival curve.

Neoadjuvant Cohort.

Two gene expression cohorts from patients treated with neoadjuvant chemotherapy totaled 311 patients (Hess et al., J Clin Oncol, 24:4236-4244, 2006; and Tabchy et al., Clin Cancer Res, 16:5351-5361, 2010). Sixty (19%) of these patients had complete pathological response. The majority of patients received paclitaxel and fluorouracil-doxorubicin-cyclophosphamide. Both datasets were examined on the same array platform (Affymetrix U133A) using a standard operating procedure, and normalization method (dCHIP) was used as previously reported (Mazouni et al., J Clin Oncol, 25:2650-2655, 2007; and Moody et al., Cancer Cell, 8:197-209, 2005). Data were downloaded from the Gene Expression Omnibus (“www.ncbi.nlm.nih.govigeo/query/acc.cgi?acc=GSE20271”) and the institutional website (“bioinformatics.mdanderson.org/pubdata.html”). Normalised expression values for both CD8 and CD68 were established as previously described (Brennan et al., Clin Cancer Res, 14:2681-2689, 2008).

TABLE 4-1
Datasets Used For The Retrospective
Gene Expression Survival Analysis

| | | | Sample | |
|---|---|---|---|---|
| Ref.* | Accession | Availability | # | Platform Type |
| S1 | GSE7849 | Processed only | 78 | Affymetrix Human Genome |
| | | | | U95 V2 Array |
| S2 | GSE3143 | Raw CEL files | 158 | Affymetrix Human Genome |
| | | | | U95 V2 Array |
| S3 | GSE10510 | Raw data | 152 | DKFZ Div of Molecular |
| | | | | Genome Analysis Human |
| | | | | Operon 4.0 olio Array 35k |
| S4 | NA | Processed only | 295 | Agilent |
| S5 | NA | Processed only | 118 | Affymetrix U133AAofAv2 |
| S6 | GSE9893 | Raw data | 155 | MLRG Human 21K V12.0 |
| S7 | GSE7390 | Raw CEL files | 198 | Affymetrix U133A |
| S8 | GSE16391 | Raw CEL files | 48 | Affymetrix U133 Plus 2.0 |
| S9 | GSE1992 | Processed only | 99 | Agilent |
| S10 | GSE4922 | Raw CEL files | 249 | Affymetrix UI33A1B |
| S11 | NA | Processed only | 69 | Agilent 44K oligo array |
| S12 | GSE9195 | Raw CEL files | 77 | Affynietrix U133 Plus 10 |
| S13 | GSE6532 | Raw CEL files | 414 | Affymetrix U133A/B and |
| | | | | plus2 |
| S14 | GSE1378, | Processed only | 60 | Custom 22K oligo array |
| | GSE 1379 | | | |
| S15 | GSE3494 | Raw CEL files | 251 | Affymetrix U133AI3 |
| S16 | GSE1456 | Raw CEL files | 159 | Affymetrix U133A/B |
| S17 | GSE21653 | Raw CEL files | 266 | Affymetrix U133 Plus 2.0 |
| S17 | GSE17907 | Raw CEL files | 51 | Affymetrix U133 Plus 2.0 |
| S18 | GSE11121 | Raw CEL files | 200 | Affymetrix U133A |
| S19 | GSE2034 | Raw CEL files | 286 | Affymetrix U133A |
| S20 | GSE12093 | Raw CEL files | 136 | Affymetrix U133A |
| S21 | GSE2109 | Raw CEL files | 353 | Affymetrix U133 Plus 2.0 |

*References: S1, Anders et al., PLoS One, 3: e1373, 2008; S2 Bild et al., Nature, 439: 353-357, 2006; S3 Calabro et al., Breast Cancer Res Treat, 116: 69-77, 2009; S4, Chang et al., PNAS, 102: 3738-3743, 2005; S5 Chin et al., Cancer cell, 10: 529-541, 2006; S6, Chanrion et al., Clin Cancer Res, 14: 1744-1752, 2008; S7, Desmedt et al., Clin Cancer Res, 13: 3207-14, 2007; S8, Desmedt et al., BMC Medical Genomics, 2: 40, 2009; S9, Hu et al., BMC Genomics, 7: 96, 2006; S10, Ivshina et al., Cancer Res, 66: 10292-1301, 2006; S11, Kok et al., Breast Cancer Res Treat, 116: 69-77, 2009; S12, Loi et al., BMC Genomics, 9: 239, 2008; S13, Loi et al., BMC Genomics, 9: 239, 2008; S14, Ma et al., Cancer Cell, 5: 607-616, 2004, S15, Miller et al., PNAS, 102: 13550-13555, 2005; S16, Pawitan et al., Breast Cancer Res, 7: R953-R9642005, 2005; S17, Sabatier et al., Breast Cancer Res Treat, 2010; S18, Schmidt et al., Cancer Res, 68: 5405-5413, 2008; S19, Wang et al., Lancet, 365: 671-679, 2006; S20, Zhang et al., Breast Cancer Res Treat, 116: 303-309, 2008; and S21, Expression Project for Oncology (expO), “expo.intgen.org/geo/home.do”

Macrophages and CD8 Infiltration Predict Survival and Chemotherapeutic Response.

Induction of an anti-tumor CD8+CTL response as a result of macrophage depletion enhances sensitivity to chemotherapy and regulates primary tumor development and development of disseminated metastases. Thus mRNA expression of CD68 and CD8 are biomarkers predictive of survival. Expression levels of CD68 and CD8 were determined from a collection of ˜4000 patients from 22 retrospective gene expression analyses. Median expression for both CD68 and CD8 was used to determine high and low groups. All patients were categorized as having either a CD68^{high}/CD8^{low} or a CD68^{low}/CD8^{high} immune signature. Kaplan-Meier analysis in the cohort (totaling ˜4000 patients) was used to determine the correlation between gene expression signatures and survival outcome.

Analysis of CD68 and CD8 Expression Levels from Previous Data Sets and Determination of Predictive Response to Chemotherapy.

Expression levels of CD68 and CD8 were determined from a cohort of 311 patients constructed from two independent data sets (Tabchy et al., Clin Cancer Res, 16: 5351-5361, 2010; and Hess et al., J Clin Oncol, 24: 4236-4244, 2006) All patients had fine needle aspirates (FNA) taken prior to neoadjuvant chemotherapy and pathological response was assessed at the time of definitive surgery. Using median expression as a threshold, examination of CD68 and CD8 mRNA in FNA samples was used to determine the correlation between expression levels and response to chemotherapy as measured by the rate of pCR in patients

### Results

Expression Levels of CD68 and CD8 are Predictive of Patient Survival Outcome.

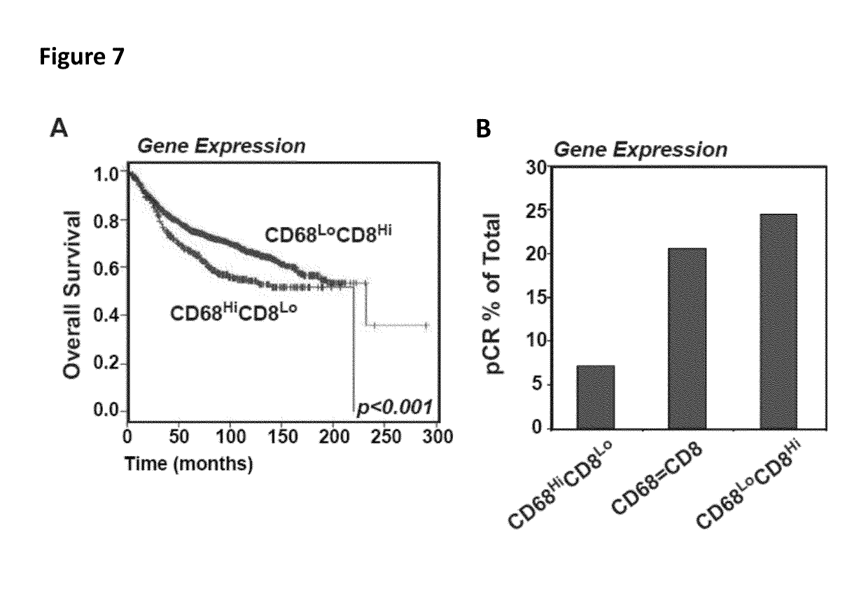

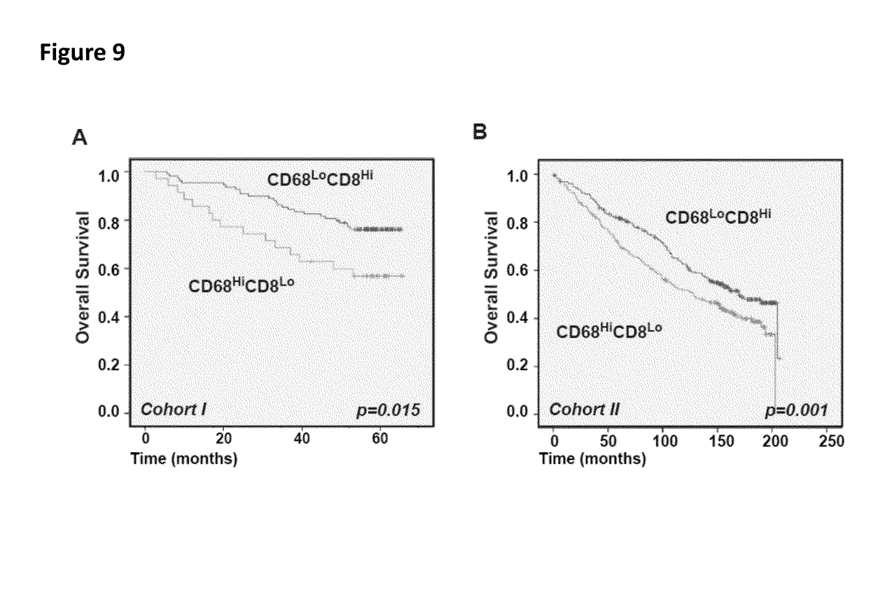

Following assignment of median expression levels into either high or low groups, all patients were categorized as having either a CD68^{high}/CD8^{low} or a CD68^{low}/CD8^{high} immune signature. Kaplan-Meier analysis in the cohort (totaling ˜4000 patients from 22 datasets) demonstrated significantly reduced survival in patients whose tumors harbored the CD68^{high}/CD8^{low} signature (FIG. 7A). These results indicate that expression levels of CD68 and CD8 are predictive of patient survival outcome, wherein an expression signature of CD68^{high}/CD8^{low} is predictive of low patient survival, and an expression signature of CD68^{low}/CD8^{high} is predictive of a higher patient survival outcome. In addition, these gene expression results were validated utilizing Kaplan-Meier analysis of OS on immunohistochemistry data from Cohort I and II stratified for CD68^{high}/CD8^{low} or CD68^{low}/CD8^{high} (FIGS. 9A and 9B).

Expression Levels of CD68 and CD8 are Predictive of Patient Response to Chemotherapy.

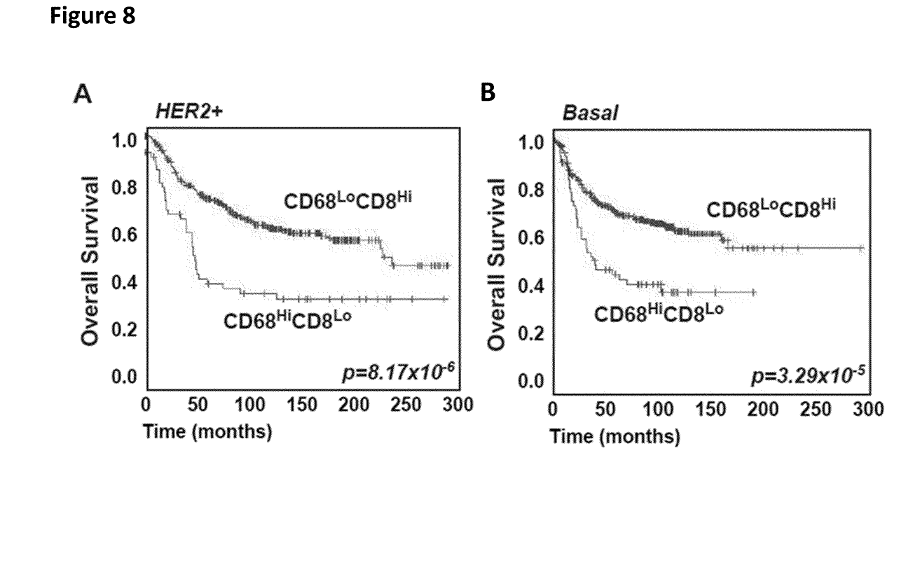

CD8 mRNA expression in FNA samples correlated with pCR (R=0.216, p<0.001), however CD68 did not. Using median expression as a threshold, examination of CD8 and CD68 mRNA in FNA samples demonstrated three specific groups CD68>CD8, CD68<CD8, and CD68=CD8 (denoted CD68^{high}/CD8^{low}, CD68/CD8^{equal} and CD68^{low}/CD8^{high} respectively). Analysis of the rate of pathological complete response (pCR) in these groups demonstrated that the CD68^{high}/CD8^{low} group had a significantly lower rate of pCR (7%) compared to the other two groups. Interestingly the CD68^{low}/CD8^{high} had the highest rate of pCR at 27% (FIG. 7B). These results indicate that expression levels of CD68 and CD8 are predictive of patient response to chemotherapy, wherein an expression signature of CD68^{high}/CD8^{low} is predictive of low pCR/poor response to neo-adjuvant chemotherapy and an expression signature of CD68^{low}/CD8^{high} is predictive of higher pCR/highest response to neo-adjuvant chemotherapy. Breast cancer is now appreciated to encompass several distinct molecular sub-types (luminal A, luminal B, HER2-positive, basal type/triple negative), possessing distinct histopathological and molecular features, correlating with differential responses to therapy and patient outcome (Perou et al., 2000; Sorlie et al., 2001; Sorlie et al., 2003). The application of breast cancer sub-typing to stratify patients and aid in treatment decisions has been proposed. To investigate if stratification of patients CD68 and CD8 expression improved risk assessment the prognostic value of the immune signature within individual tumor subtypes was analyzed. This analysis revealed that the CD68^{high}/CH₈^{low} phenotype was associated with reduced OS in both HER2⁺ and basal tumor subtypes (FIGS. 8A and 8B), but not luminal A and B tumors. This correlation in basal disease is particularly important given the aggressive nature of basal (and/or triple-negative) breast cancers that can become refractory to chemotherapy.

### Example 5

### Antibody-Based Method for Determining the Immune Signature of Tumor-Infiltrating Leukocytes in Pancreatic Ductal Adenocarcinoma Patients

This example describes methods for immunophenotyping tumor-infiltrating leukocytes, and provides results of immune signature analyses in a cohort of pancreatic ductal adenocarcinoma patients (PDAC) patients.

Materials and Methods

Patients and Tumor Samples.

Tissue microarray (TMA) studies were conducted on a patient cohort, constructed from 59 cases of invasive PDAC diagnosed at the Department of Pathology, Washington University (St. Louis, Mo.). The cohort of PDAC patients were eligible for surgery with no detectable metastasis at the time the tissue sample was collected. Patients had not received neo-adjuvant therapy and were treated with pancreaticoduodenectomy surgical resection typically followed by adjuvant chemotherapy. To assemble TMAs, clearly defined areas of tumor tissue were indicated on a slide with a fresh tissue section from the paraffin block. Two biopsies, 1.0 mm in diameter, were taken from each donor paraffin block corresponding to the marked area. In general, cores were taken from the peripheral aspect of the tumor. Necrotic tissue was avoided. For immunohistochemical analyses, 4.0 mm paraffin sections were used.

Immunohistochemistry.

Tissue microarray slide sections were prepared and immunohistochemistry was performed as described in Example 1.

Automated Image Acquisition, Management, and Analysis.

Fully automated image acquisition was employed. The Aperio ScanScope XT Slide Scanner (Aperio Technologies, Vista, Calif.) system was used to capture whole slide digital images with a 20× objective. Slides were de-arrayed to visualize individual cores, using Spectrum software (Aperio). A tumor specific nuclear algorithm (IHC-MARK) developed in house was modified to quantify CD8 and CD68 expression. IHC-MARK was initially designed to identify tumor cells on the basis on nuclear morphology (Rexhapaj et al., Cancer Res, 10:R89, 2008), however this was modified for evaluating leukocyte infiltration based on specific nuclear morphology.

Results

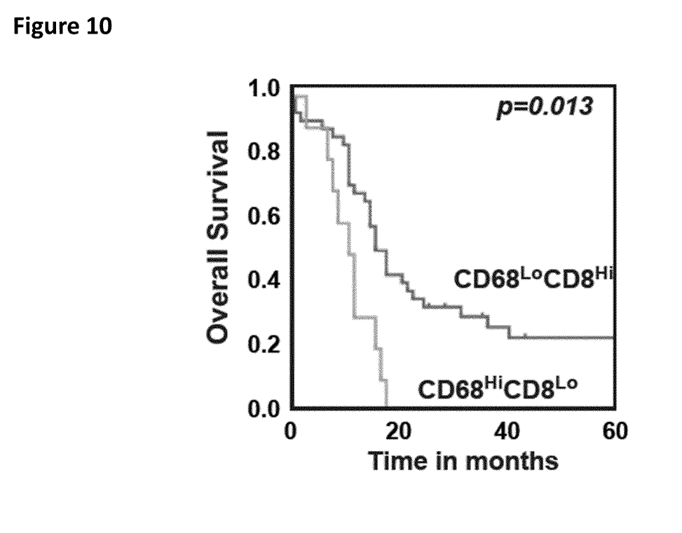

To understand if the alterations in tumor infiltrating immune cells might be indicative of altered survival in PDAC patients, a tissue microarray containing tumor tissue from 59 patients was analyzed. Tumors were scored for the presence of CD68⁺ and CD8⁺ leukocytes and stratified into two groups (CD68^{Hi}/CD8^{Lo} and CD68^{Lo}/CD8^{Hi}). In patients where CD68⁺ macrophages were the dominant tumor infiltrating leukocyte (CD68^{Hi}/CD8^{Lo}), overall survival was significantly reduced as compared to the other group (denoted as CD68^{Lo}/CD8^{Hi}). A Kaplan-Meier estimate of overall survival is shown in FIG. 10.

Various modifications and variations of the present disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are understood by those skilled in the art are intended to be within the scope of the claims.

We claim:

1. A method for assessing risk of poor clinical outcome for a human pancreatic cancer patient, said method comprising:
a) subjecting a sample from a pancreatic tumor of said patient to a procedure for quantitation of expression of leukocyte biomarkers comprising CD8 and CD68;
b) detecting the presence of an immune signature of poor outcome comprising CD8^{lo},CD68^{hi} or an immune signature of favorable outcome comprising CD8^{hi},CD68^{lo} in said sample, wherein said immune signature of poor outcome is associated with an increased risk of poor clinical outcome as compared to said immune signature of favorable outcome, and wherein said biomarkers do not comprise CD4.

2. The method of claim 1, wherein said poor clinical outcome comprises a relative reduction in one or more of overall survival, recurrence-free survival, and distant recurrence-free survival.

3. The method of claim 1, further comprising:
c) treating said patient with a palliative treatment regimen when said immune signature of poor outcome is detected.

4. The method of claim 3, wherein said palliative treatment regimen comprises a surgical resection with palliative intent selected from the group consisting gastric bypass, biliary bypass, and biliary stent.

5. The method of claim 1, wherein said favorable clinical outcome comprises a relative increase in one or more of overall survival, recurrence-free survival, and distant recurrence-free survival.

6. The method of claim 1, further comprising:
c) treating said patient with a curative treatment regimen when said immune signature of favorable outcome is detected.

7. The method of claim 6, wherein said curative treatment regimen comprises a radical pancreatic resection.

8. The method of claim 7, further comprising post-operative chemotherapy comprising one or more of gemcitabine, fluorouracil, erlotinib and mitomycin c.

9. The method of claim 1, further comprising:
a step before a) of obtaining said sample of said pancreatic tumor from said patient.

10. The method of claim 1, wherein said pancreatic tumor is a pancreatic ductal adenocarcinoma.

11. The method of claim 1, wherein said procedure for quantitation is an antibody-based technique.

12. The method of claim 11, wherein said antibody-based technique comprises immunohistochemistry.

13. The method of claim 1, wherein said procedure for quantitation is a nucleic acid-based technique.

14. The method of claim 13, wherein said nucleic acid-based technique comprises reverse transcriptase-polymerase chain reaction or gene expression array.

15. The method of claim 1, further comprising detecting metastasis to a regional or a draining lymph node of said human cancer patient.
